(19) United States
(12) Patent Application Publication
(10) Pub. No.: US 2013/0302787 A1
(43) Pub. Date: Nov. 14, 2013

(54) CARTRIDGE FOR USE IN AN AUTOMATED SYSTEM FOR ISOLATING AN ANALYTE FROM A SAMPLE, AND METHODS OF USE

(71) Applicants: NORTHWESTERN UNIVERSITY, Evanston, IL (US); QUIDEL CORPORATION, San Diego, CA (US); NORTHWESTERN GLOBAL HEALTH FOUNDATION, Evanston, IL (US)

(72) Inventors: Abhishek K. Agarwal, Evanston, IL (US); Renana Ashkenazi, Evanston, IL (US); Mark J. Fisher, Highland Park, IL (US); Paul J. Gleason, Laguna Niguel, CA (US); Jacqueline R. Groves, Chicago, IL (US); Henry H. Hsu, Aliso Viejo, CA (US); David M. Kelso, Wilmette, IL (US); Sally M. McFall, Evanston, IL (US); Mark E. Mossberg, Santa Ana, CA (US); Zaheer Parpia, Evanston, IL (US); Kunal Sur, Evanston, IL (US); Tom Westberg, Gurnee, IL (US)

(73) Assignees: Northwestern University, Evanston, IL (US); Northwestern Global Health Foundation, Evanston, IL (US); Quidel Corporation, San Diego, CA (US)

(21) Appl. No.: 13/888,951

(22) Filed: May 7, 2013

Related U.S. Application Data
(60) Provisional application No. 61/644,387, filed on May 8, 2012.
(60) Provisional application No. 61/774,392, filed on Mar. 7, 2013.

(51) Int. Cl. C12N 15/10

(52) U.S. Cl. CPC C12N 15/1006
USPC 435/5; 435/309.1; 422/559

(57) ABSTRACT

A device for extraction or isolation of an analyte, such as a nucleic acid, a protein, or a cell, from a sample, and in particular from a biological sample, is described. Methods of using the device are also described. Further processes, such as amplification of the isolated analyte, may also be carried out within the device.

### CROSS-REFERENCE TO RELATED APPLICATIONS

This application claims the benefit of U.S. Provisional Application No. 61/644,387, filed May 8, 2012 and of U.S. Provisional Application No. 61/774,392, filed Mar. 7, 2013, each of which is incorporated herein by reference in its entirety.

### TECHNICAL FIELD

The subject matter described herein relates generally to a cartridge device useful for extraction or isolation of an analyte, such as a nucleic acid, a protein, or a cell, from a sample, and in particular from a biological sample.

### BACKGROUND

Effective analysis of biological entities, such as proteins or nucleic acids, in biological samples generally requires that the target entity in question first be isolated from the biological matrix, which frequently includes a complex mixture of non-target substances. The effective isolation of analytes is a prerequisite for efficient downstream analysis of the analyte, including, for example, amplification of a nucleic acid for detection and quantification. It is also important, in many cases, such as in nucleic acid amplification, that the isolated species not contain residues of certain reagents and/or solvents used during isolation.

Existing methods of isolation frequently involve multistep processes, often requiring multiple extraction and/or centrifugation steps, which require trained personnel and can introduce risks of contamination and/or loss of sample. A need exists for a self-contained device that is effective to isolate an analyte from a biological sample, such as obtained from a patient, with minimal operator manipulation of sample and reagents.

While automated or modular systems are available, e.g. for conducting protein or nucleic acid separation, immunoassays, and nucleic acid amplification, their cost and complexity often limits their usefulness in smaller laboratories and clinics, particularly in developing nations. There is an increasing need for low-cost, rapid and reliable diagnosis and monitoring of diseases such as HIV, tuberculosis, and pertussis in the developing world. To this end, self-contained devices effective to isolate an analyte from a biological sample obtained from a patient, with minimal operator input, would be of great use, particularly if the device was also effective to carry out the analysis.

### BRIEF SUMMARY

The following aspects and embodiments thereof described and illustrated below are meant to be exemplary and illustrative, not limiting in scope.

Disclosed herein, in one aspect, is a sample processing device, comprising a rigid body having a first side and a second side, and defining at least a first cavity, a second cavity, and a third cavity wherein the first, second and third cavities are associated with first, second, and third storage compartments, respectively, each containing a water-miscible liquid reagent. The device also comprises a first channel, connecting the first cavity and the second cavity, and a second channel region, in fluid communication with and downstream of the second cavity, and connected to the third cavity via a third channel, at a first intersection, wherein the second channel region is associated with a storage compartment containing a water-immiscible fluid. A wall member is secured to at least a portion of the first side of the rigid body, the wall member disposed over the first cavity, the second cavity, and the third cavity, thereby defining a first chamber, a second chamber, and a third chamber. An inlet port is in direct communication with the first chamber; and a plurality of solid carrier particles are optionally provided in the first chamber.

Preferably, the second channel region in the device is in communication with the first channel and first cavity only via the second chamber.

In a preferred embodiment, the storage compartment containing a water-immiscible fluid contains a volume of the fluid that is sufficient, when dispensed to the second channel region from the storage compartment, to produce a continuous layer of the fluid within the second channel region that includes the first intersection.

The device may include further chambers, such as a fourth chamber, which is in communication with the second channel region via a second intersection, upstream of the first intersection, and which is associated with a fourth liquid reagent storage compartment, containing a water-miscible reagent. In this case, the storage compartment containing a water-immiscible fluid preferably contains a volume of the fluid that is sufficient, when dispensed to the second channel region from the storage compartment, to produce a continuous layer of the fluid within the second channel region that includes the first and second intersections. The fourth storage compartment may contain an aqueous or aqueous ethanolic solution.

The device may also include a fifth chamber, which is in communication with the second channel region, upstream of the third chamber. In this case, the storage compartment containing a water-immiscible fluid may contain a volume of the fluid that is sufficient, when dispensed to the second channel region from the storage compartment, to fill at least a portion of the fifth chamber and to produce a continuous layer of the fluid within the second channel region that includes the first and second intersections. Preferably, the fifth chamber is in communication with the second channel region either at the first intersection or at a third intersection which is upstream of the first intersection.

In one embodiment, the wall member of the device comprises a plurality of blister regions defining the liquid reagent storage compartments. Alternatively, the device may comprise a blister layer which comprises a plurality of blister regions defining the liquid reagent storage compartments.

In selected embodiments, the water-miscible liquid reagent in each of the first, second and third storage compartments is selected from an aqueous buffer, a water-containing lysis buffer, a water-based salt solution, and an elution medium.

The second storage compartment may contain a volume of liquid reagent that is greater than the combined volume of the second chamber and any intermediary conduit. Alternatively, it may contain at least a volume of liquid reagent effective to fill the second chamber, the first channel and any intermediary conduit.

The device may comprises a conduit connecting the second storage compartment to the first channel, or to a region of the first chamber immediately adjacent the first channel, or to the second chamber.

In another embodiment, the third storage compartment contains a volume of liquid reagent that is greater than the combined volume of the third chamber, the third channel and any intermediary conduit.

In one embodiment, the third chamber comprises optically transparent windows which make up a portion of the exterior surface of the rigid body.

Preferably, the device comprise at least one mixing member in at least one of the first chamber and the second chamber; the mixing member may be, for example, a stir bar, a mixing ball, and/or a series of raised ridges in a cavity or a channel of the rigid body.

Preferably, the plurality of solid carrier particles comprises a plurality of magnetic particles. One or more of the particles is typically treated on its external surface with an affinity reagent capable of associating with an analyte. The affinity reagent may be, for example, an antibody or antibody fragment with specific binding for an analyte, such as a protein, or a nucleic acid sequence capable of hybridizing with an analyte

Also disclosed herein, in a related aspect, is a method for extracting an analyte of interest from a sample, comprising (i) providing a device comprising: a first chamber, containing a solid phase carrier and comprising a sample port, a second chamber, and a third chamber which is a process chamber, a first channel, connecting the first chamber and the second chamber, and a second channel region, in fluid communication with and downstream of the second chamber, and connected to the third chamber via a third channel, at a first intersection; (ii) introducing into the first chamber, a volume of a first aqueous reagent, and the sample, wherein the solid phase carrier is effective to selectively bind the analyte if present in the sample; (iii) introducing a volume of a second aqueous reagent into the second chamber, effective to fill the second chamber and at least a portion of the first channel; and introducing a volume of a third aqueous reagent into the third chamber and third channel; (iv) introducing a volume of water-immiscible fluid into the second channel region, such that the water-immiscible fluid forms a contiguous zone of fluid within the second channel region that includes the first intersection, and forms first and second fluid interfaces, respectively, with the second aqueous reagent and with the third aqueous reagent; and (vi) with an externally applied force, moving the solid phase carrier, sequentially, into the aqueous reagent in the second chamber, into the water-immiscible fluid, and into the third aqueous reagent in the third channel and processing chamber The moving transfers the solid phase carrier and associated analyte of interest, thereby extracting the analyte of interest from the sample.

Preferably, the water-miscible/water-immiscible fluid interfaces formed by introduction of the water-immiscible fluid remain essentially stationary during the moving of the solid phase carrier.

In another preferred embodiment, the second channel region of the device is in communication with the first channel and first cavity only via the second cavity.

The device may further comprise a fourth chamber, which is in fluid communication with the second channel region at a point upstream of the first intersection. In this case, the method may further comprise, subsequent to step (ii) and prior to step (iv): introducing into the fourth chamber a fourth aqueous reagent, which forms a further fluid interface with the water-immiscible fluid within the second channel region, and the moving may comprise: moving the solid phase carrier, sequentially, into the aqueous reagent in the second chamber, into the water-immiscible fluid, into the aqueous reagent in the second chamber, into the water-immiscible fluid, into the third channel, and into the third aqueous reagent in the third channel and processing chamber.

The device may further comprise a drying chamber, which is connected to the second channel region at a point at or upstream of the first intersection, wherein the method further comprises, prior to moving the solid phase carrier into the third channel and processing chamber, moving the solid phase carrier into the drying chamber, and subsequently filling at least the portion of the drying chamber containing the solid phase carrier with the water-immiscible fluid.

Preferably, the plurality of solid carrier particles comprises a plurality of magnetic particles. One or more of the particles is typically treated on its external surface with an affinity reagent capable of associating with an analyte. The affinity reagent may be, for example, an antibody or antibody fragment with specific binding for an analyte, such as a protein, or a nucleic acid sequence capable of hybridizing with an analyte. The analyte of interest may be, for example, a protein or a nucleic acid. When the analyte of interest is a nucleic acid, the method may further comprise amplifying the nucleic acid within the third (process) chamber.

In selected embodiments, the water-miscible liquid reagent in each of the first, second and third storage compartments is selected from an aqueous buffer, a water-containing lysis buffer, a water-based salt solution, and an elution medium. When the sample contains cells, the reagent introduced into the first chamber preferably comprises a cell lysis reagent.

In one embodiment, useful for achieving a contiguous volume of process chamber reagent within the device that is known and precise, the volume of reagent introduced into the process chamber is greater than the volume of the process chamber, such that an excess portion of the process chamber reagent flows into a channel in communication with and upstream of the process chamber; and subsequent introduction of the water-immiscible fluid is effective to displace the excess portion of the process chamber reagent at a predetermined location, which may be at the second fluid interface noted above. The excess portion of the process chamber reagent may be transferred into an upstream chamber, such as the fourth chamber, or a further chamber, situated between the third and fourth chambers and in communication with the second channel region, into which the transferred portion can flow.

In another embodiment, the first channel includes a constriction region having a dimension, and a divider having a first height; the second channel region includes a divider having a second height which is greater than the first height; the volume of aqueous reagent introduced into the second chamber is effective to fill the second chamber and the first channel, to a level above the first divider but below the second divider; and the combined volume of aqueous reagent introduced into the first and second chambers is effective to fill the first and second chambers and the first channel, to a level above the first divider but below the second divider.

In a related embodiment, the solid phase carrier comprises a plurality of solid carrier particles, and the number of particles in the plurality of solid carrier particles, the size of each particle in the plurality of solid carrier particles, and the dimension of the constriction region are selected such that the plurality of solid carrier particles individually and collectively can pass through the constriction region; and the constriction region reduces transfer of aqueous reagent via the first channel between the first and second chambers.

In addition to the exemplary aspects and embodiments described above, further aspects and embodiments will become apparent by reference to the drawings and by study of the following descriptions.

Additional embodiments of the present devices and methods, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIG. 1 shows one embodiment of a cartridge device as disclosed herein, in exploded view;

FIGS. 2 and 4 are side views of the device of FIG. 1, containing various liquid reagents, represented by shading, at different stages of addition;

FIG. 3 is a three-dimensional side view of a further embodiment of a cartridge device as disclosed herein, with liquid reagents represented by shading;

FIGS. 5A-5C show another embodiment of a cartridge device, where FIG. 5A shows a front view, FIG. 5B shows a back view of the body without a wall member attached, and FIG. 5C shows a back view with the wall member and storage chambers attached;

FIGS. 6A-6B show the front and back surfaces (without a front cover film) of a cartridge device as disclosed herein, in accordance with another embodiment;

FIG. 7 shows the device of FIGS. 6A-6B, in exploded view;

FIG. 8 is a detail view of cavities (chambers), channels and conduits within the body of the device of FIGS. 6A-6B; and

FIGS. 9 and 10 are detail views of the device of FIGS. 6A-6B, containing various liquid reagents, represented by shading, at different stages of addition.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

It must be noted that, as used in this specification, the singular forms “a,” “an,” and “the” include plural referents unless the context clearly dictates otherwise.

A “liquid reagent”, as the term is used herein, refers to any liquid contained within any of the storage compartments of the cartridge device as described herein, including aqueous, nonaqueous, and water-immiscible liquids.

A “reagent solution” typically refers to an aqueous solution. The “reagent” in a reagent solution may be a chemical or biological substance that causes a chemical change to a sample component, or it may be simply a buffering agent, a salt, or a solvent.

A region within a sample processing device, such as a cavity, chamber, or channel, is “in communication with” or “in fluid communication with” another such region if there is a continuous path between the two regions, such that liquid could be (but not necessary is) transferred between them. In some cases, a valve or seal must be opened before such transfer occurs.

A storage compartment is “associated with” a respective chamber or channel when the two are connected via one or more conduits, channels, and/or ports, such that the contents of the compartment can be transferred to the chamber or channel. Typically, seals or valves are provided to prevent premature transfer of contents.

A “specific binding member” or “affinity reagent”, as used herein, is a molecule or moiety that specifically binds to a target analyte through chemical or physical means. Immunoreactive specific binding members include antigens or antigen fragments and antibodies or functional antibody fragments. Other specific binding pairs include biotin and avidin, carbohydrates and lectins, complementary nucleotide sequences, effector and receptor molecules, cofactors and enzymes, enzyme inhibitors and enzymes, and the like.

In the extraction/isolation procedures described herein, a binding member is attached to a solid phase support, such as a plurality of paramagnetic particles, in order to extract the analyte from a sample containing non-target components. Following isolation of the particle-analyte complex from the non-target components, the complex is treated to effect removal of the analyte from the particles. Removal may be effected by, for example, heating the solution containing the complex and/or changing the chemical environment (e.g. salt concentration, pH, etc.). In other embodiments, a chemical or enzymatic reagent is used to disrupt the particle-analyte complex and thus effect removal of the analyte from the particles.

Particular examples of systems designed for formation of specific particle-analyte complexes and their subsequent release of analyte include, for example, the MagneHis™ protein purification system (Promega Corp., Madison, Wis.), in which paramagnetic precharged nickel particles (MagneHis™ Ni-Particles) are used to isolate polyhistidine- or HQ-tagged proteins from a sample matrix such as a cell lysate. Also preferred are functionalized solid supports as described in U.S. Pat. No. 7,354,750 (D. J. Simpson et al., Promega Corp.). Alternatively, the MagneGST™ protein purification system (Promega Corp.) employs immobilized glutathione paramagnetic particles (MagneGST™ Particles) to isolate glutathione-S-transferase (GST) fusion proteins. In the HaloTag® protein purification system (Promega Corp.), useful for purification of recombinant proteins, the protein of interest is expressed as a fusion protein, fused to a HaloTag® protein tag, which covalently binds to a HaloLink™ solid support via an immobilized chloroalkane ligand. Following separation of the fusion protein-resin complex from other matrix components, a specific protease then cleaves the target protein from the fused tag and the resin. The protease is also tagged such that it will remain bound to the resin.

An “isolated” analyte is one that has been separated from other constituents with which it is associated in a sample, such that it can be detected with a desired degree of accuracy and precision. The isolated analyte is typically dissolved in a solvent medium that may also contain non-interfering substances. In the case of a biological sample, the analyte is isolated from cellular constituents with which it is normally associated, and from other types of cells which may be present in the sample.

### II. Cartridge Device

Disclosed herein, in one aspect, is a device useful for extraction of an analyte of interest from a matrix containing the analyte, such as a biological sample. The analyte could be, as described further below, a protein, a nucleic acid, or a cell or cell component. In other embodiments, the sample could be an environmental sample.

The cartridge device is particularly useful for automated extraction, and preferably automated analysis as well, where only minimal operator input is required, when employed in conjunction with an instrument such as described further below.

In general, a preferred sample processing device comprises a rigid body having a first side and a second side, and defining at least a first cavity, a second cavity, and a third cavity, wherein the first, second and third cavities are associated with first, second, and third storage compartments, respectively, each containing a water-miscible liquid reagent. The device also comprises a first channel, connecting the first cavity and the second cavity, and a second channel region, in fluid communication with and downstream of the second cavity, and connected to the third cavity via a third channel, at a first intersection, wherein the second channel region is associated with a storage compartment containing a water-immiscible fluid, a wall member secured to at least a portion of the first side of the rigid body, said wall member disposed over the first cavity, the second cavity, and the third cavity, thereby defining a first chamber, a second chamber, and a third chamber, which may be a lysis chamber, wash chamber, and elution/process chamber, respectively. An inlet port is in communication, direct or indirect, with the first chamber; and a plurality of solid carrier particles are optionally present in the first chamber.

Preferably, the second channel region is in communication with the first channel and first cavity only via said second chamber. As discussed further below, little or no actual fluid transfer takes place between the second channel region and the first channel and first cavity/chamber.

The storage compartment containing a water-immiscible fluid preferably contains a volume of said fluid that is sufficient, when dispensed to the second channel region from the storage compartment, to produce a continuous layer of the water-immiscible fluid within the second channel region that includes the first intersection.

In certain embodiments, the device further comprises a fourth chamber, which may be a further wash chamber, in communication with the second channel region via a second intersection, upstream of the first intersection. This chamber is associated with a fourth liquid reagent storage compartment, containing a water-miscible reagent.

In this case, the storage compartment containing a water-immiscible fluid preferably contains a volume of said fluid that is sufficient, when dispensed to the second channel region from the storage compartment, to produce a continuous layer of the water-immiscible fluid within the second channel region that includes the first and second intersections.

The device may further comprise a fifth chamber, which may be a drying chamber, in communication with said second channel region, upstream of the third chamber. The fifth chamber may be in communication with the second channel region either at the first intersection described above, or at a third intersection which is upstream of the first intersection.

The storage compartment containing a water-immiscible fluid preferably contains a volume of said fluid that is sufficient, when dispensed to the second channel region from the storage compartment, to produce a continuous layer of the water-immiscible fluid within the second channel region that includes a portion of the fifth chamber, and which includes the first and second intersections; that is, in operation, at least the first and second intersections and the region between them will contain the water-immiscible fluid, and at least a portion of the fifth chamber may contain the water-immiscible fluid.

The liquid reagent storage compartments may be defined by a plurality of blister regions; these may be contained within the wall member mentioned above, or they may be contained within a separate blister layer. The storage compartments, in one embodiment, are attached to the outer, external surface of the rigid body of the device, and can be integrally formed with the wall member of the device, which can be of a flexible material, such as a foil laminate. That is, the storage compartments are not integrally formed with the rigid body, but are externally attached to the rigid body.

Preferably, the water-miscible liquid reagent in each of the first, second and third storage compartments is selected from an aqueous buffer, a water-containing lysis buffer, a water-based salt solution, and an elution medium. The fourth storage compartment may contain an aqueous or aqueous ethanolic solution.

The second storage compartment preferably contains a volume of liquid reagent that is greater than the combined volume of the second chamber (and any intermediary conduit); more preferably, the second storage compartment contains at least a volume of liquid reagent effective to fill said second chamber and said first channel (and any intermediary conduit). It may also contain at least a volume of liquid reagent effective to fill the second chamber, the first channel, a portion of the second channel region, and/or a portion of said first chamber (and any intermediary conduit).

In one embodiment, the second storage compartment is connected to the first channel, preferably adjacent the first channel; in other embodiments, it is connected to the second chamber. It may also be connected to a region of the first chamber that is immediately adjacent the first channel.

In selected embodiments, the third storage compartment contains a volume of liquid reagent that is greater than the combined volume of the third chamber and the third channel (and any intermediary conduit).

Other preferred features of the device will be set forth in the more detailed descriptions below.

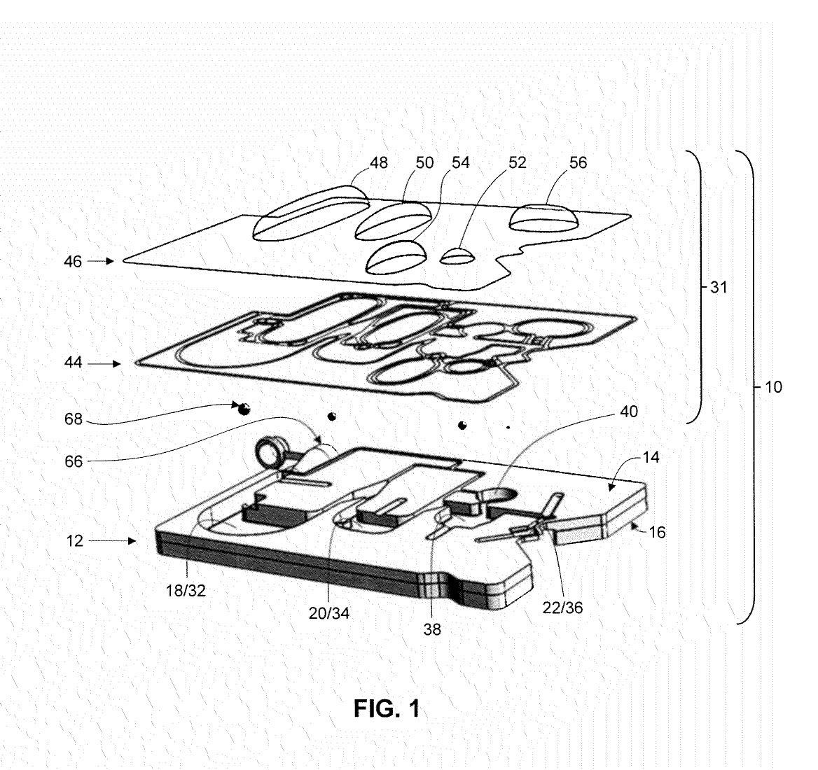

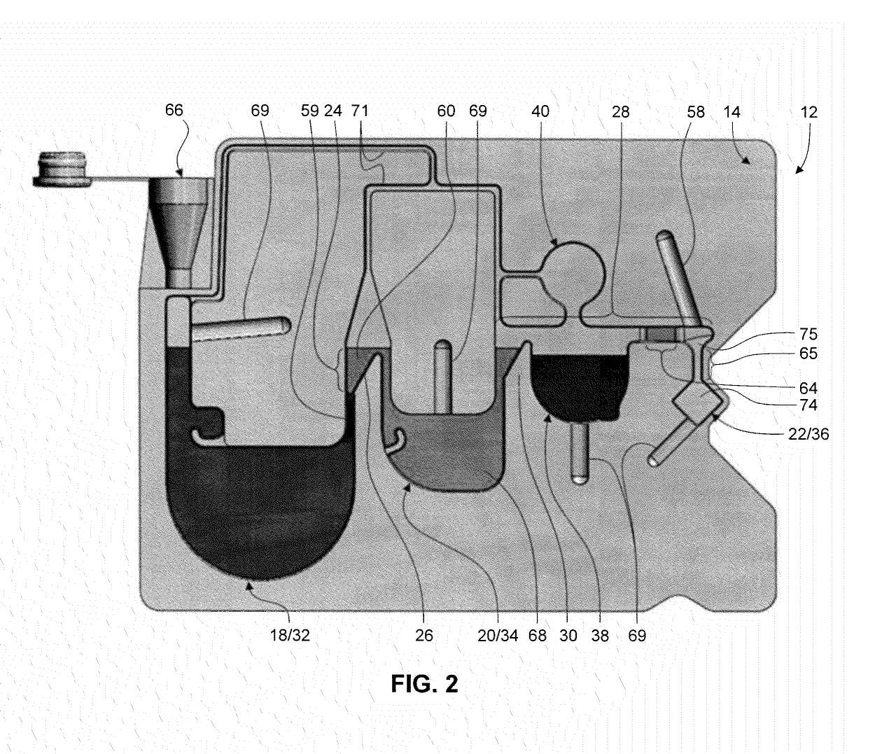

One embodiment of the device (which may be referred to as the “horizontal” format) is shown in an exploded view in FIG. 1. FIG. 2 shows the body of the device, in this embodiment, in greater detail. As shown therein, the device 10 comprises a rigid body 12 having a first side 14 and a second side 16. Preferably, the device is designed to be used in an upright position as shown in the Figures. The body 12 is molded or otherwise fabricated to define, at least, a first cavity 18, a second cavity 20, and a third cavity 22. With reference to FIG. 2, a first channel 24 connects the first cavity 18 and the second cavity 20. Separating the first cavity 18 and the second cavity 20 is a first divider 26 having a first specified height. In one embodiment, and as shown in FIG. 1, the first divider has a sloped wall to form a tapered channel on one side of the divider. As seen channel 24 is a tapered channel by virtue of the sloped wall in divider 26. A second channel region 28 connects the second cavity 20 and the third cavity 22. Between the second cavity 20 and the third cavity 22 is a second divider 30, which has a height greater than that of the first divider 26 (FIG. 2).

The device further comprises, as shown in FIG. 1, a wall member 31 secured to at least a portion of the first side 14 of the rigid body 12. The wall is disposed over the various cavities to form respective chambers, e.g. a first chamber 32, a second chamber 34, and a third chamber 36. (Chambers are indicated by reference numbers in FIGS. 2-4, even though, for the sake of clarity, the wall member is not shown in these Figures.) The disclosure herein is directed to the rigid body 12 both individually and in combination with the wall unit 31.

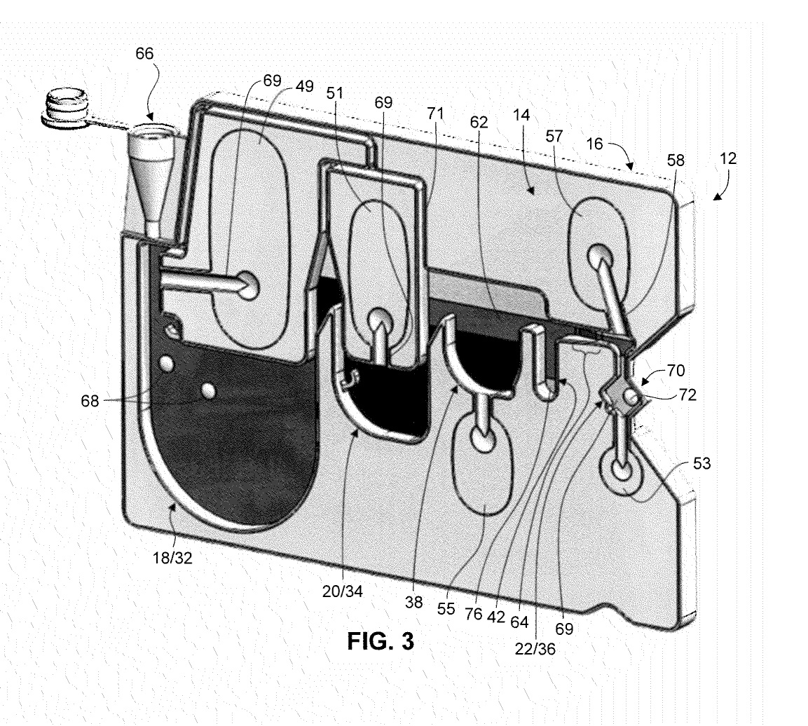

The device may include further chambers in addition to those described above, and in addition to those illustrated. For example, in selected embodiments, the device includes a fourth cavity and chamber, such as shown at 38, in fluid communication with second channel region 28. The device may also include a fifth cavity and chamber, as shown at 40, disposed above second channel region 28. FIG. 3 illustrates an embodiment containing a further chamber 42 in fluid communication with the second channel region, upstream of third chamber 36.

The wall member 31 disposed over the various cavities to form the various chambers may comprise, as shown in FIG. 1, a penetrable sealing layer 44 and a layer 46 comprising one or more blister regions, defining one or more liquid reagent storage compartments. Sealing layer 44 may comprise foil or other thin flexible material that seals the blister regions to create the storage compartments and is secured to the rigid body 12.

Each storage compartment is typically associated with a chamber within body 12. By “associated with” is meant that the compartment and respective chamber are connected via a conduit and/or port, such that the contents of the compartment can be transferred to the chamber. Seals or valves are generally provided to prevent premature dispensing of reagents. For example, in the embodiment of FIG. 1, blister regions defining storage compartments 48, 50, 52 and 54 are associated with chambers 32, 34, 36, and 38, respectively. (The reference numbers which indicate the blister regions in FIG. 1 are also used to refer to the sealed storage compartments.) These storage compartments correspond to footprint regions 49, 51, 53, and 55, respectively, in FIG. 3 (not shown in FIG. 1 or 2).

A further blister region 56 defines a liquid reagent storage compartment which is associated with region 57 and channel 58, which is in fluid communication with the second channel region 28, as shown in FIGS. 2 and 3.

The wall member, and more particularly the penetrable sealing layer 44, comprises an opening or an openable surface associated with each of the liquid reagent storage compartments, through which liquid reagent can flow from the storage compartment to the associated chamber and/or channel in the device. Fluid flow could also be controlled by valves.

In particular embodiments, the storage compartments have particular capacities relative to the chambers in the device, as disclosed below. When the components are assembled into device 10, as provided to the user, some or all of the storage compartments will contain liquid reagents, as disclosed herein.

Each of the liquid reagent storage compartments is able to deliver a liquid reagent to an associated chamber, either directly or indirectly via an associated conduit. In particular embodiments, the liquid reagent to be contained in each of storage compartments 48, 50, 52 and 54 is a water-miscible liquid reagent, preferably selected from an aqueous buffer, a water-containing lysis buffer, a water-based salt solution, a water-alcohol solution, and an elution medium. In selected embodiments, as discussed further below, storage compartment 48, associated with first chamber 32, contains a water-containing lysis buffer; storage compartment 50, associated with second chamber 34, contains an aqueous wash buffer; and storage compartment 52, associated with third chamber 36, contains an elution medium. Storage compartment 54, associated with fourth chamber 38, may contain a water-alcohol solution. In one embodiment, storage compartment 54 contains a non-alcohol aqueous medium or is empty.

Preferably, the storage compartment 56 comprises a water-immiscible substance, as described further below.

A further preferred feature of the device of FIGS. 1-4 is a constriction region 59 (FIG. 2), having a specified dimension, situated in channel 24 between first chamber 32 and second chamber 34. The constriction region has a small cross sectional area and is effective to reduce, minimize and/or substantially prevent transfer or mixing of fluid, via the first channel, between the first and second chambers. In selected embodiments, the diameter or width of the constricted area is about 5 mm or less, more preferably about 2.5 mm or less, and most preferably about 1 mm or less. In some embodiments, the diameter or width of the constriction region may be about 0.5 mm, about 0.3 mm, or about 0.1 mm or less. Preferably, the diameter or width of the constriction region is at least 0.1 mm, and more preferably at least 0.5 mm.

Also provided is a solid phase carrier, preferably a plurality of solid carrier particles (not shown in the Figures), which are added to, or preferably provided within, the first chamber 32. The solid carrier particles are able to pass through the chambers and channel s upon application of an external force. In one embodiment, the particles are magnetic particles, and the external force is a magnetic force.

Preferably, the size of the particles and the dimension of the constriction region 59 are selected such that the plurality of solid carrier particles can pass individually and collectively through the constriction region upon application of the external force. Commercially available magnetic particles employed in the biomedical field typically range in size from less than 1 micron up to 100 microns, most commonly in the size range of 2-10 microns; preferably, the particles employed herein are about 1-3 microns in size.

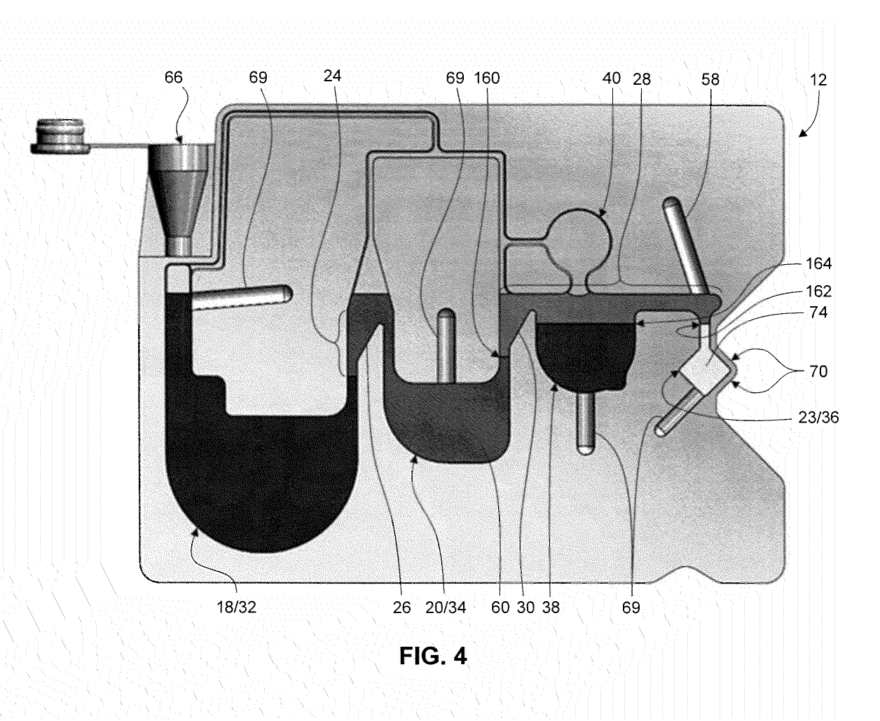

The storage compartments (48, 50, 52, 54, 56) are effective to deliver selected volumes of liquid reagents to the respective chambers. For example, the second storage compartment 50 (FIG. 1; footprint 51 in FIG. 3) preferably has a volume effective to hold a volume of liquid reagent that is greater than the volume of second chamber 34 (plus the volume of any intermediary conduit) and thereby may contain and deliver a volume of liquid reagent that is greater than the volume of second chamber 34. By “greater than the volume of the second chamber” is meant that a portion of the liquid reagent 60 delivered to second chamber 34 preferably flows over the top of the first divider 26, to at least partially fill the region between the top of the first divider 26 and the constriction region 59, as shown, for example, in FIGS. 2 and 4. However, the liquid reagent 60 does not flow over second divider 30, which is higher than first divider 26 (FIG. 2).

In a further preferred embodiment, the combined volume of liquid reagent contained in the first storage compartment 48 and the second storage compartment 50 is effective to fill the first and second chambers 32 and 34 to a level above the first divider 26 but below the second divider 30 (FIG. 2). In addition, the volume of liquid reagent contained in the first storage compartment 48 is such that it would fill first chamber 32 to a level below the first divider 26 if the liquid reagent 60 were not present.

In further preferred embodiments, the third chamber 36 has a volume less than the volume of the first chamber 32, and preferably less than the volume of the second chamber 34. Preferably, for reasons discussed further below, the third storage compartment 52 (FIG. 1; footprint 53 in FIG. 3) can contain a volume of liquid reagent that is greater than the volume of third chamber 36 (plus the volume of any intermediary conduit). More preferably, the third storage compartment 52 can contain a volume of liquid reagent that is greater than the combined volume of third chamber 36 and connected channel 23 (FIG. 2) (plus the volume of any intermediary conduit).

The liquid reagent storage compartment 56 (footprint 57 in FIG. 3) preferably comprises a volume of water-immiscible fluid substance, as described further below, that is sufficient to fill the second channel region 28, via conduit 58, creating a contiguous layer 62 of water-immiscible fluid substance that reaches from the third chamber to the second chamber, including the second divider, as shown, for example, in FIGS. 3 and 4.

The device also includes, as shown in the figures, an inlet port 66 in fluid communication (direct or indirect) with at least the first chamber. Mixing members such as 68 (FIGS. 1 and 3) may be included in any of the chambers, and are preferably included in at least the first and second chambers. The mixing member(s) may comprise stir bar(s) or mixing ball(s), which can be magnetically activated from outside of the device. Alternatively, the mixing member(s) may comprise one or more series of raised ridges (“washboards”) in one or more cavity walls and/or within one or more channels of the rigid body. Preferably, these ridges are arranged within the cavities and/or channels and have a dimension such that each particle must pass over the ridges in being transported through the cavities and/or channels.

As shown in the Figures, connecting conduits 69 for reagent delivery and air channels 71 for pressure equalization are also provided within rigid body 12.

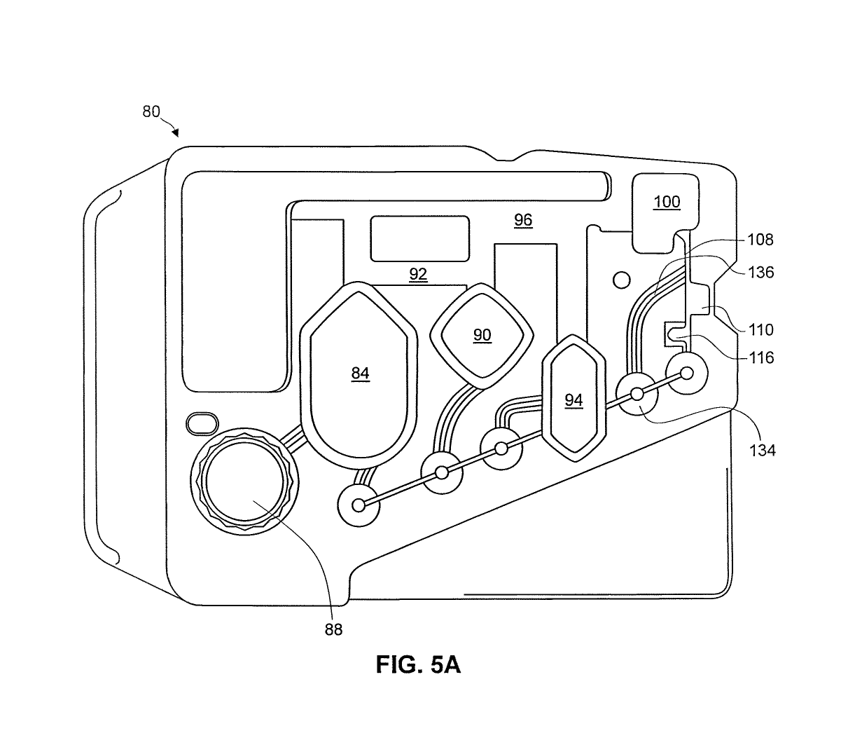

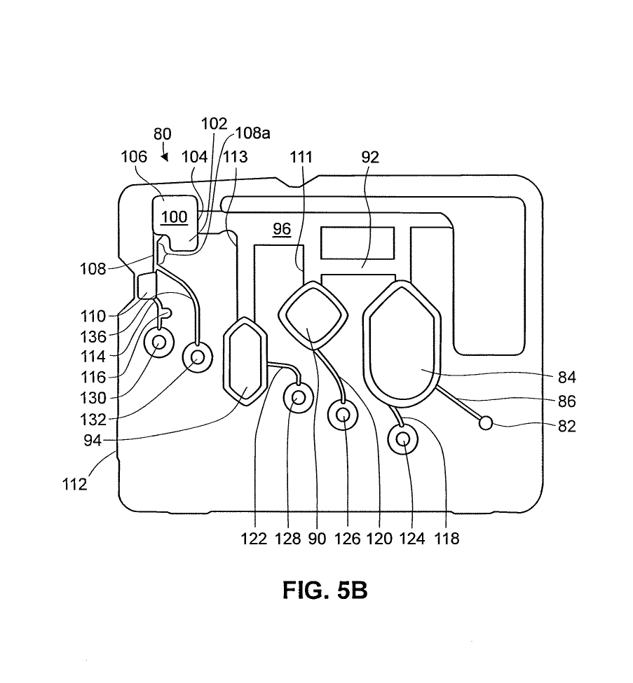

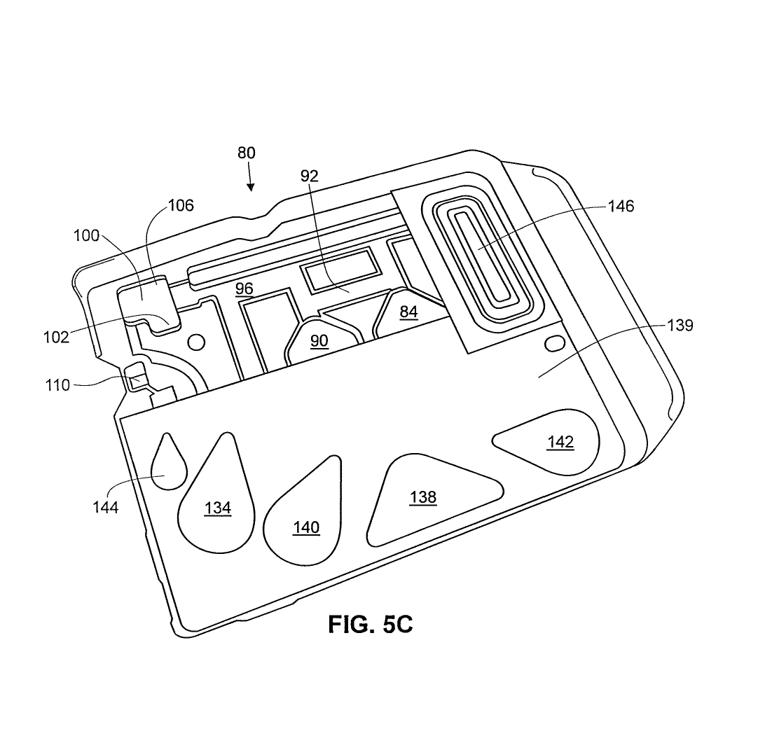

Optionally, the device includes a narrowed region 64 of the second channel region, as shown in FIGS. 3 and 5, between second divider 30 and third chamber 36, and preferably adjacent to channel 23. As shown in FIG. 3, the narrowed region of the channel region is between the third chamber 36 and any other chamber connected to the second channel region. Following the narrowed region is channel 23, which may be used as an elution region, to release captured analytes from supports prior to entering chamber 36.

The device also includes, associated with third chamber 36, optical windows 70 (FIG. 3) for detection of optical signals, e.g. for monitoring the progress of an amplification reaction. The region of the device containing third chamber 36 is accessible to heating, for example by placement within an instrument having suitably disposed heating elements, for use in e.g. thermal cycling processes. Other regions of the device, particularly the region associated with first chamber 32, may also be accessible to heating in a similar manner. The cross-sectional width of the body 12 may also be less in the area of the third chamber, also referred as the processing chamber, 36, as shown, for example, in FIG. 3, to improve heat transfer in this region.

Additional reagents to be employed within third chamber 36 may be included within the chamber, typically in lyophilized form, within a wax pellet 72, as shown in FIG. 3, which can be melted to release the reagents at an appropriate time.

The assembled device 10 is designed for automated use within a instrument that may hold one or a plurality of such devices, as described further below. Accordingly, the device 10 may contain external features, such as notches or ridges, used to properly align the device within the instrument.

Another device embodiment is shown in FIGS. 5A-5C. Cartridge 80 is shown in a front view in FIG. 5A and is made of a rigid material in which a plurality of cavities and conduits can be formed. A back view of the cartridge is seen in FIG. 5B. A sample entry port 82 permits a user to introduce a sample into a first cavity or chamber 84 of the cartridge. Entry port 82 is in fluid connection with first chamber 84 by a conduit 86. As seen in FIG. 5A, entry port 82 may have a cap 88 to open and close the entry port from the external environment.

Cartridge 80 additionally comprises a second chamber 90 in fluid communication with first chamber 84 via channel or conduit 92. A third chamber 94 is in fluid communication with the second chamber 90 via a channel 96. Channel 96 is also in fluid communication with a fourth chamber 100, which has a lower portion 102 positioned below the opening 104 where channel 96 terminates into chamber 100 and an upper portion 106 above opening 104. Chamber 100 is in fluid communication via conduit 108 with a fifth chamber 110. Fifth chamber is also referred to as a processing chamber, and is situated along an edge 112 of cartridge 80 for optical inspection of the contents in chamber 110.

Chamber 100 is a dual purpose chamber. Lower portion 102 is dimensioned to receive and contain excess fluid (overfill) from processing chamber 110. As described below, in some embodiments a precise amount of fluid in processing chamber is desired for reaction control. A precise amount of fluid is provided by overfilling chamber 110 so that fluid enters conduit 108. When an immiscible fluid is introduced into the cartridge also as described below, the overfill processing chamber fluid in conduit 108 is displaced into the lower portion 102 of chamber 100. Chamber 100 in its upper portion 106 provides an air gap for pressure equalization and for movement of the particle-analyte complexes into the air gap to permit removal of volatile solvents or other liquid reagents from the complexes prior to transfer of the complexes into the processing chamber.

Conduit 108 comprises a narrow portion or region of construction 108a in the flow path processing chamber 110 and its adjacent chamber. The constriction region provides fluid control as the chambers are filled with fluid from the storage compartments and required the particle-analyte complexes to separate somewhat from adjacent particle-analyte complexes to assist in removal of fluid from the plurality of particles as the plurality is moved through the conduit.

Device 80 also comprises a first dividing wall 111 that has a first height and a second dividing wall 113 that has a second height greater than the first dividing wall. This feature also provides for control of fluids during filling of the chambers and conduits of the device, and minimizes undesired mixing of fluids in each respective chamber of the device.

A conduit 114 is in communication with processing chamber 110, and in this embodiment conduit 114 includes a holding chamber 116. Holding chamber 116 is dimensioned and positioned to receive and contain the plurality of particles. For example, detection or amplification of an analyte in processing chamber 110 may proceed optimally in the absence of the plurality of particles. In this case, the analyte can be eluted from the particles and the particles moved by the externally applied force into the holding chamber. The analyte to be processed and/or detected remains in the processing chamber.

Each chamber 84, 90, and 94 has an associated reagent conduit, such as conduits 118, 120 and 122, respectively. Conduit 114 serves as regent conduit for the processing chamber 110. Each of conduits 114, 118, 120 and 122 is associated with an opening, seen best in FIG. 5B, as openings 124, 126, 128 and 130. Each opening is associated with a storage compartment, seen best in FIG. 5C, that contains a liquid or liquid reagent that can be introduced via the opening into a respective chamber.

Opening 132 and its associated conduit 136 are in communication with a storage compartment 134 filled with an immiscible fluid. The immiscible fluid flows from the storage compartment via opening 132 into conduit 136, displacing processing reagent fluid in conduit 108 into the lower portion 102 of chamber 100. The immiscible fluid flows via opening 104 into conduit 96 and, if desired, into conduit 92. In some embodiments, conduit 92 is filled with a buffer or wash solution, introduced via opening 126 and conduit 120 from an associated storage compartment 138 that holds sufficient solution to fill conduit 120, chamber 90 and conduit 92.

With reference to FIG. 5C, the back side of cartridge 80 is shown, where a wall member 139 is placed over the rigid body, enclosing the cavities and conduits formed therein to define chambers and channels. The wall member comprises a plurality of storage chambers, preferably integrally formed with the wall member, wherein each storage chamber contains a fluid that is dispensed into its associated chamber during use of the cartridge. As mentioned above, storage compartment 134 contains an immiscible fluid, and is in fluid communication via opening 132 and conduit 136 with the flow path in channels 108 and 96. Storage compartment 138 is in fluid communication via opening 126 and conduit 120 with chamber 90. Storage compartment 140 is in fluid communication via opening 128 and conduit 122 with chamber 94, and storage compartment 142 is in fluid communication via opening 124 and conduit 118 with chamber 84. A storage compartment 144 is filled with a fluid for use in the processing chamber 110, and is provided to the processing chamber via port 130 and conduit 114.

Wall member 139 may also comprises an inflatable member, such as member 146. Inflatable member 146 is positioned over an air vent or an air collection zone in the cartridge, and can inflate as needed to accommodate air from the chambers and channels in the cartridge that is displaced when fluid from the storage chambers is dispensed into the cartridge.

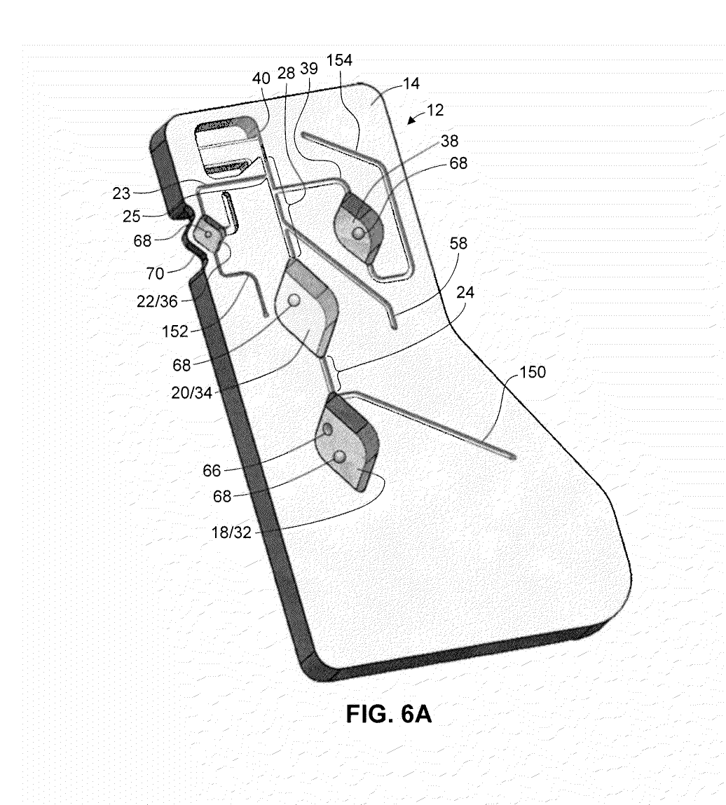

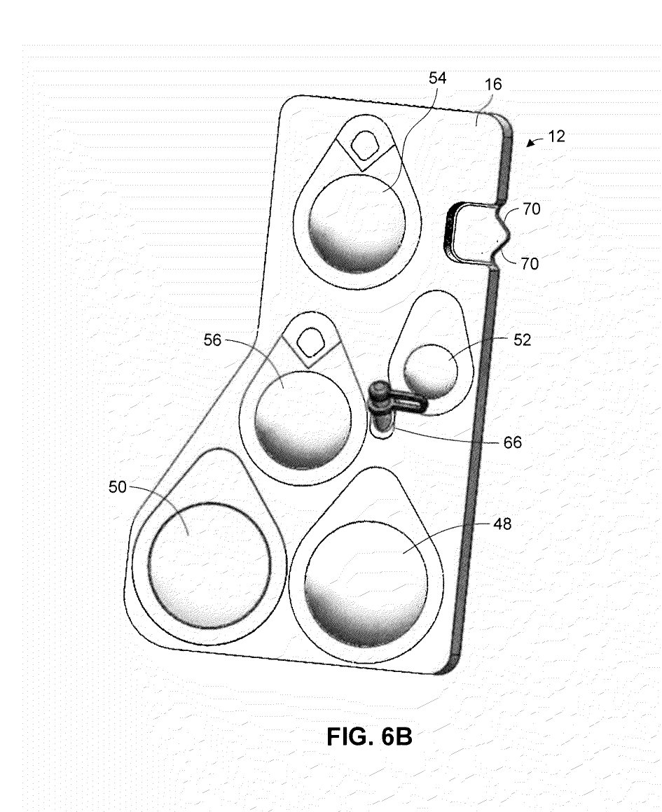

FIGS. 6-10 show an alternative design of a cartridge device, which may be referred to as the “vertical” format, with the respective chambers and storage compartments indicated using the numerical identifiers of FIGS. 1-4 to indicate similar cartridge features.

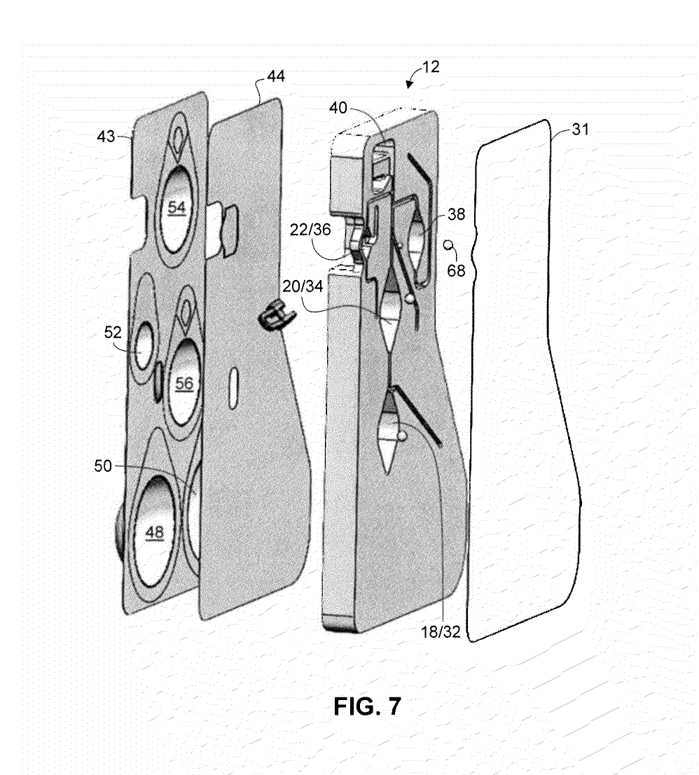

With reference to FIGS. 6-7, where FIG. 7 in shown in exploded view, the device 10 comprises a rigid body 12 having a first “front” side 14 (FIG. 6A) and a second “back” side 16 (FIG. 6B). Preferably, the device is designed to be used in an upright position as shown in the Figures. The body 12 is molded or otherwise fabricated to define, at least, a first cavity 18, a second cavity 20, and a third cavity 22 (FIG. 6A; FIG. 7), as in the horizontal format described above. With reference to FIG. 6A, a first channel 24 connects the first cavity 18 and the second cavity 20. A second channel region 28 is downstream and in communication with the second cavity 20, and is connected to third cavity 22, via channel 23, at first intersection 25.

The device further comprises, as shown in FIG. 7, a wall member 31, such as a cover film (not shown in FIG. 6 for reasons of clarity), secured to at least a portion of the first side 14 of the rigid body 12. The wall is disposed over the various cavities to form respective chambers, e.g. a first chamber 32, a second chamber 34, and a third chamber 36. (Chamber reference numbers are included in FIG. 6 for the purpose of illustration, even though the cover film is not shown in the Figure.)

The device may include further chambers in addition to those described above, and in addition to those illustrated. For example, in selected embodiments, the device includes a fourth cavity and chamber, such as shown at 38, in fluid communication with second channel region 28. In this embodiment, the chamber is in communication with second channel region 28 via conduit 39.

The device may also include a fifth cavity and chamber, as shown at 40, disposed in communication with second channel region 28, upstream of third chamber 36. The chamber may be connected to the second channel region either at the same intersection (25) as channel 23, as shown in FIG. 6A, or at a further intersection (not illustrated) upstream of intersection 25. For reasons described below, chamber 40 may contain a plurality of compartments having different depths with respect to front face 14, as depicted in FIG. 6A.

The device is also provided with one or more vents as required for fluid movement in filling the chambers and channels and/or with one or more drains for removal of excess fluid. These may be present, for example, in the fifth cavity described above.

As shown in FIG. 7, the “back” side of the device is adapted to comprise one or more blister regions, e.g. in blister layer 43, defining one or more liquid reagent storage compartments. Sealing layer 44 may comprise foil or other thin flexible and pierceable material that seals the blister regions to create the storage compartments and is secured to the rigid body 12. Fluid flow could also be controlled by valves.

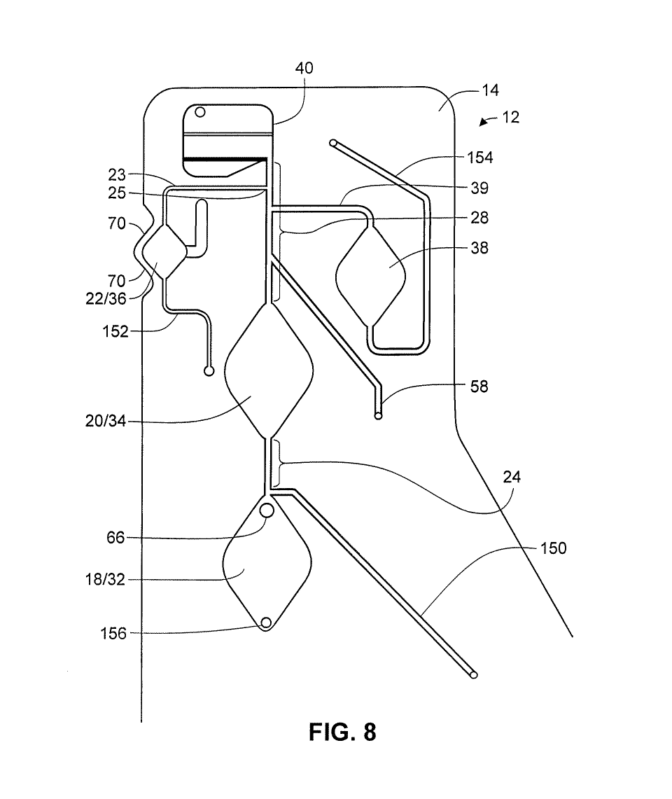

Each storage compartment is typically associated with a chamber within body 12. By “associated with” is meant that the compartment and respective chamber are connected via one ore more conduits, channels, and/or ports, such that the contents of the compartment can be transferred to the chamber. Seals or valves are generally provided to prevent premature dispensing of reagents. For example, blister regions defining storage compartments 48, 50, 52 and 54, as shown in the embodiment of FIG. 6B, are associated with chambers 32, 34, 36, and 38, respectively. Storage compartments 50, 52 and 54 are connected to their respective chambers via conduits 150, 152, and 154, respectively. In the case of conduit 150, it may be connected directly to the second chamber; or to the first channel, preferably adjacent to the first chamber, as shown in FIG. 8; or to a region of the first chamber immediately adjacent the first channel, as shown in FIG. 6A.

Ports at the termini of the conduits, in main body 12, provide access to the storage compartments. In the embodiment shown in FIG. 8, storage compartment 48 communicates with chamber 32 via a port 156. Chamber 32 also has a sample entry port 66.

Mixing members 68, such as described above, may be included in any of the chambers, and are preferably included in at least the first and second chambers.

A further blister region 56 (FIG. 6B) defines a liquid reagent storage compartment which is associated with channel region 28, via conduit 58, as shown in FIG. 6A.

The penetrable sealing layer (blister cover) 44 comprises an opening or openable surface associated with each of the liquid reagent storage compartments, through which liquid reagent can flow from the storage compartment, though suitably located ports provided in main body 12, into the associated chamber and/or channel in the device. Fluid flow could also be controlled by valves.

In particular embodiments, the storage compartments have particular capacities relative to the chambers in the device, as disclosed below. When the components are assembled into device 10, as provided to the user, some or all of the storage compartments will contain liquid reagents, as disclosed herein.

Each of the liquid reagent storage compartments is able to deliver a liquid reagent to an associated chamber, either directly, via a port, or via an associated conduit. In particular embodiments, the liquid reagent to be contained in each of storage compartments 48, 50, 52 and 54 is a water-miscible liquid reagent, preferably selected from an aqueous buffer, a water-containing lysis buffer, a water-based salt solution, a water-alcohol solution, and an elution medium. In selected embodiments, as discussed further below, storage compartment 48, associated with first chamber 32, contains a water-containing lysis buffer; storage compartment 50, associated with second chamber 34, contains an aqueous wash buffer; and storage compartment 52, associated with third chamber 36, contains an elution medium. Storage compartment 54, associated with fourth chamber 38, may contain a water-alcohol solution. In one embodiment, storage compartment 54 contains a non-alcohol aqueous medium or is empty.

Preferably, the storage compartment 56 comprises a water-immiscible substance, as described above.

Also provided is a solid phase carrier, preferably a plurality of solid carrier particles (not shown in the Figures), which are added to, or preferably provided within, the first chamber 32. The solid carrier particles, as described above, are able to pass through the chambers and channels upon application of an external force. In one embodiment, the particles are magnetic particles, and the external force is a magnetic force.

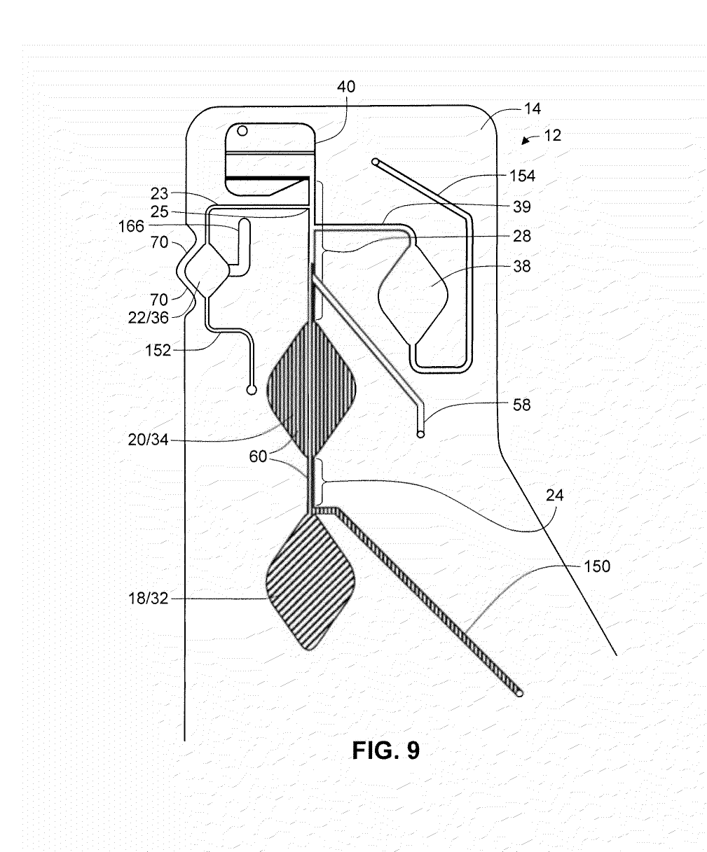

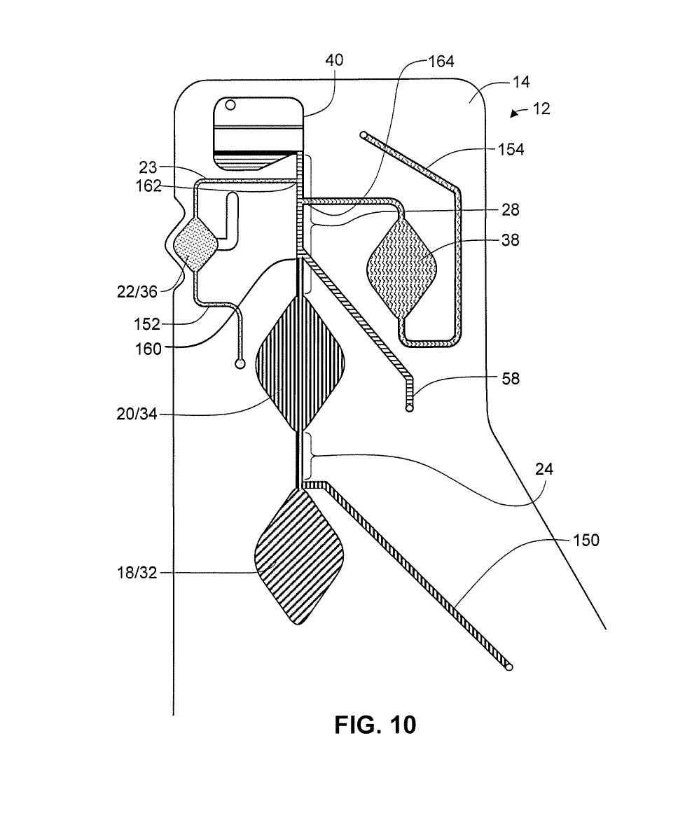

The storage compartments (48, 50, 52, 54, 56) are effective to deliver selected volumes of liquid reagents to the respective chambers. For example, the second storage compartment 50 preferably has a volume effective to hold a volume of liquid reagent that is greater than the volume of second chamber 34 (plus the volume of any intermediary conduit) and thereby may contain and deliver a volume of liquid reagent, via conduit 150, that is greater than the volume of second chamber 34. By “greater than the volume of the second chamber” is meant that the volume of the liquid reagent 60 (see FIGS. 9-10; horizontal hatching) is effective to fill second chamber 34 and preferably a portion of second channel region 28 immediately adjacent to second chamber 34 (as shown, for example, in FIGS. 9-10) (in addition to the volume of conduit 150 itself). The volume of liquid reagent 60 may also be sufficient to fill a small portion of first chamber 32, particularly when conduit 150 is connected to a region of the first chamber immediately adjacent the first channel, as shown in FIG. 6A.

The liquid reagent storage compartment 56 preferably comprises a volume of water-immiscible fluid, as described above, that is sufficient to fill the second channel region 28, via conduit 58, creating a contiguous body 62 (as shown in FIG. 10; vertical hatching) of water-immiscible fluid substance that extends from the junction of conduit 58 with the second channel region 28 to include at least first intersection 25 with third channel 23. The volume of water-immiscible fluid may also be sufficient to further fill one or more portions of chamber 40.

The device also includes, associated with third chamber 36, optical windows 70 (see e.g. FIG. 6) for detection of optical signals, e.g. for monitoring the progress of an amplification reaction. The region of the device containing third chamber 36 is accessible to heating, for example by placement within an instrument having suitably disposed heating elements, for use in e.g. thermal cycling processes. Other regions of the device, particularly the region associated with first chamber 32, may also be accessible to heating in a similar manner. The cross-sectional width of the body 12 may also be less in the area of process chamber 36, as shown, for example, in FIG. 7, to improve heat transfer in this region.

### III. Methods of Use

The devices described herein are useful for isolation of target substances from biological samples and, preferably, for detection and/or quantification of the isolated substances (analytes).

Preferably, the biological sample is first introduced into a first chamber of the device via an inlet port. Depending on the nature of the sample, it may be pretreated in various ways, e.g. by dilution with a standard buffer, if necessary.

Liquid reagents are introduced into the chambers from the associated storage compartments, preferably in a preselected and automated sequence. The selection of liquid reagents and the sequence in which they are added will depend on the process to be carried out within the device. Chambers may include, for example, reagents for isolation, separation, modification, labeling, and/or detection of analytes. Reagents may be added simultaneously and/or in sequence.

In general, a method for extracting an analyte of interest from a sample using the devices described herein comprises (i) providing a device as described above. The device preferably comprises a first chamber containing a solid phase carrier (although it will be appreciated that the solid phase carrier can also be introduced into the device by a user) and comprising a sample port, a second chamber, and a third chamber, which is a processing chamber. A first channel, connecting the first chamber and the second chamber, and a second channel region, in fluid communication with and downstream of the second chamber, and connected to the third chamber via a third channel, at a first intersection are also present in the device. The method comprises introducing into the first chamber, a volume of a first aqueous reagent, and the sample. In one embodiment, the sample a solid phase carrier are introduced into the first chamber; in another embodiment, the solid phase carrier is present in the first chamber, and the sample is introduced. The solid phase carrier is effective to selectively bind the analyte if it is present in the sample.

Then, a volume of a second aqueous reagent is introduced into the second chamber. The volume is effective to fill the second chamber and at least a portion of the first channel. Then, a volume of a third aqueous reagent is introduced into the third chamber and third channel. A volume of water-immiscible fluid is introduced into the second channel region, such that the water-immiscible fluid forms a contiguous zone of fluid within the second channel region that includes the first intersection, and forms separate fluid interfaces with the second aqueous reagent and with the third aqueous reagent. With an externally applied force, the solid phase carrier is moved, sequentially, into the aqueous reagent in the second chamber, into the water-immiscible fluid, and into the third aqueous reagent in the third channel and processing chamber. Moving transfers the solid phase carrier and associated analyte of interest, thereby extracting the analyte of interest from the sample.

Preferably, the separate fluid interfaces remain essentially stationary during the moving of the solid phase carrier.

In a preferred embodiment, the second channel region is in communication with the first channel and first cavity only via the second chamber. Most preferably, as noted below, little or no fluid transfer occurs between the second channel region and the first channel and first cavity/chamber.

In some embodiments, in which the device further comprises a fourth chamber, which is in fluid communication with the second channel region at a point upstream of the first intersection, the method further comprises, introducing into the fourth chamber a fourth aqueous reagent, which forms a further fluid interface with the water-immiscible fluid within the second channel region, and the moving comprises moving the solid phase carrier, sequentially, into the aqueous reagent in the second chamber, into the water-immiscible fluid, into the aqueous reagent in the second chamber, into the water-immiscible fluid, into the third channel, and into the third aqueous reagent in the third channel and processing chamber.

Again, all of the water-miscible/water-immiscible fluid interfaces, formed when the fluids are dispensed into the chambers and channels in accordance with the disclosed method, preferably remain essentially stationary when the solid carrier particles are moved through the device, in a manner to be described below. In essence, these fluid interfaces preferably remain fully stationary, with the exception of minor disturbances that may be caused by the movement of the particles themselves through the interfaces.

In other embodiments, in which the device further comprises a drying chamber, which is connected to the second channel region at a point at or upstream of the first intersection,

the method further comprises, prior to moving the solid phase carrier into the third channel and processing chamber, moving the solid phase carrier into the drying chamber, and subsequently filling at least the portion of the drying chamber containing the solid phase carrier with the water-immiscible fluid.

Other features of the method will be set forth in the more detailed descriptions below. In one embodiment, the method is carried out using a device such as illustrated in FIGS. 1-4.

In a preferred embodiment, a lysis buffer, effective to lyse cells in a biological sample, is introduced, from storage compartment 48 (FIG. 1; footprint 49 in FIG. 3), into first chamber 32. As described above, the volume of liquid reagent, in this case lysis buffer, contained in storage compartment 48 is such that it fills first chamber 32 to a level below the first divider 26.

Subsequently, in an exemplary process sequence, a wash buffer 60 is then introduced, from storage compartment 50 (FIG. 1; footprint 51 in FIG. 3), into second chamber 34. As shown in FIG. 2 and as described above, storage compartment 50 delivers a volume of liquid reagent, in this case wash buffer, that is greater than the volume of second chamber 34, such that a portion of the liquid reagent 60 flows over the top of the first divider 26, to at least partially fill the region between the top of the first divider 26 and the constriction region 59, as shown, for example, in FIG. 2. However, the liquid reagent 60 does not flow over second divider 30, which is higher than first divider 26; the combined volume of liquid reagent contained in the first storage compartment 48 and the second storage compartment 50 is effective to fill the first and second chambers 32 and 34 to a level above the first divider 26 but below the second divider 30.

As noted above, the region of the first channel between the first and second chambers includes a constricted region, e.g. 59, to minimize mixing of fluids between these two chambers. In addition, the second channel region is in communication with the first channel and first cavity only via the second cavity. Accordingly, minimal fluid from the first chamber (lysis buffer in one embodiment) enters the first channel, even less enters the second chamber, and virtually none contacts the second channel region, which will eventually contain a layer of water-immiscible fluid 62.

This design has advantages such as the following. It has been found that, when particles containing bound analyte freshly extracted from a lysis mixture in chamber 38 are washed in wash chamber 40 prior to being introduced to water-immiscible fluid in flow path 32, there is less tendency for the particles to clump and/or to stick to the walls of the chamber(s) and flow path(s), as compared to when particles containing bound analyte freshly extracted from lysis mixture in chamber 38 are directly introduced to the water-immiscible fluid.

Simultaneously with, subsequent to, or prior to addition of the lysis buffer and the wash buffer 60, an elution and/or reaction buffer, in a preferred embodiment, is added to third chamber (process chamber) 36 from storage compartment 52 (footprint 53 in FIG. 3). Preferably, an alcohol/water or aqueous wash solution is added to chamber 38 from storage compartment 54 (footprint 55 in FIG. 3), either simultaneously with, subsequent to, or prior to addition of the wash buffer 60, to give the arrangement exemplified in FIG. 2.

As noted above, the third storage compartment 52 preferably delivers a volume of liquid reagent 74 that is greater than the volume of third chamber 36 (process chamber), for the purpose of precisely defining the amount of fluid in the chamber. For some processes, such as nucleic acid amplification, it is necessary or highly desirable to know the precise amount of liquid reagent in the chamber. Delivery of a precise volume directly from the storage compartments can be subject to error in this respect. Thus, delivery of a precise volume to chamber 36 is achieved by first overfilling the chamber 36 with the liquid reagent (e.g. elution and/or amplification buffer), and preferably also overfilling adjacent channel 23, as shown at 75 in FIG. 2. Subsequently, and subsequent to the placement of fluids in the first and second chambers and preferably the fourth chamber, a water-immiscible fluid is introduced from storage compartment 56, via channel 58, such that the water-immiscible fluid overlays chamber 36 and displaces the overfill volume, e.g. to an upstream chamber, thereby precisely defining the volume of fluid in chamber 36 and adjacent channel 23 to their known machined volume, terminating at interface 92.

In a preferred embodiment, as shown in FIG. 3, the channel just upstream of chamber 46 contains a narrowed region 64. Narrowing the channel aids in managing the oil (or other water-immiscible fluid) front as it flows into the channel from storage compartment 57. By virtue of increased surface tension, the oil forms a plug which displaces the surplus elution buffer, rather than flowing past it. In the embodiment of FIG. 3, the surplus elution buffer, shown at 76, is displaced to a dedicated overflow chamber 42; in the embodiment of FIGS. 2 and 4, the surplus buffer (not shown) is displaced to chamber 38.

The amount of water-immiscible fluid introduced is sufficient to fill the second channel region 28, creating a contiguous layer 62 of water-immiscible substance that reaches from the top of the third chamber to adjacent the proximal outlet of the second chamber 34, including the second divider 30, as shown, for example, in FIG. 4. (It should be noted that an amount of water-immiscible fluid 62 sufficient to fill the second channel region 28 in this manner is introduced, subsequent to introduction of the remaining liquid reagents, regardless of whether the above-described strategy is used to obtain a precise amount of fluid in chamber 36.)

Accordingly, fluid interfaces (between water-miscible and water-immiscible fluid) are created at 160 and 162, with the second aqueous reagent and third aqueous reagent, respectively, as shown in FIG. 4. In a preferred embodiment, where fourth chamber 38 and its associated conduit 39 also contain a water-immiscible reagent, a further fluid interface is formed at 164 (FIG. 4). Preferably, all of these fluid interfaces, formed when the fluids are dispensed into the chambers and channels in accordance with the disclosed method, remain essentially stationary when the solid carrier particles are moved through the device, in a manner to be described below.

At some stage before, during or after the addition of fluid reagents, a plurality of affinity-treated particles (not shown in the Figures) is added to first chamber 32. The device may also be provided to the user, in a preferred embodiment, with the particles already in place in the first chamber. At least a plurality and preferably all of the particles comprise an attached specific binding member, as described above, which is effective to specifically and reversibly bind the target analyte(s); e.g. by specific antibody-antigen binding, by hybridization, by ionic or hydrogen bonding, or other chemical interaction. The binding moiety may be, for example, a nucleic acid probe sequence, effective to hybridize to a target nucleic acid sequence, or an antibody or functional fragment thereof, effective to bind a target protein or other analyte. Any binding moiety of any desired specificity may be used.

The particle-bound analyte is then exposed to the various liquid reagents within the device by a process in which the particles are moved, by virtue of an externally applied force, though the chambers and channels. Thus, following the disposition of fluids into the respective chambers and channels, to give the arrangement shown, for example, in FIG. 4, there is preferably minimal transport of fluid within the device. Preferably, all of the water-miscible/water-immiscible fluid interfaces, formed when the fluids are dispensed into the chambers and channels in accordance with the disclosed method, remain essentially stationary when the solid carrier particles are moved through the device.

Preferably, the particles are paramagnetic particles, such that they can be moved through the chambers and channels via an externally applied magnetic force. However, other means of moving the particles via an externally applied force can be used, including air pressure, vacuum, centrifugal force, or electrical fields for charged molecules or particles.

In a preferred embodiment, the sample is admixed with lysis buffer and affinity-treated particles in first chamber 32, for a sufficient time, at a sufficient temperature, and with sufficient agitation to lyse cells and allow the target analyte, if present, to bind to the affinity-treated particles. As noted above, the external sides of the device corresponding to first chamber 32 are accessible to a heat source if required, and mixing elements such as stir bars, stir particles, or “washboard” surfaces are preferably provided within the chamber. Mixing may also be facilitated by moving the particles within the chamber by the above-referenced externally applied force.

Following lysis and binding, in the exemplified process, the particles are transported, by application of the external force, to the second chamber 34, containing, in the present scenario, a wash buffer, such as a tris hydrochloride (HCl) buffer or phosphate buffered saline (PBS). Accordingly, the particles are moved through constriction 59 in the first channel, which minimizes transfer of fluid from the first chamber to the second chamber. The constriction is of narrow diameter for this purpose, but it is of sufficient diameter to allow a plurality of moderately clumped particles to pass therethrough. Commercially available magnetic particles employed in the biomedical field range in size from less than 1 micron up to 100 microns, most commonly in the size range of 2-10 micron; preferably, the particles employed herein are about 1-3 microns in size. The constricted area is preferably about 5 mm or less, more preferably about 2.5 mm or less, and most preferably about 1 mm or less in diameter or width. In some embodiments, the constriction may be about 0.5 mm, about 0.3 mm, or about 0.1 mm or less in diameter or width. Preferably, the constriction is at least 0.1 mm in diameter or width, and more preferably at least 0.5 mm in diameter or width.

Upon entering the second chamber 34, the solution therein and/or the particles may be further agitated, using one or more agitation strategies as described for the first chamber.

The particles are then moved into the layer of water-immiscible fluid 62 present in the second channel region 28. As described in U.S. Patent Appn. Pubn. No. 2009/0246782, which is incorporated herein by reference in its entiriety, movement of the carrier particles into the water-immiscible fluid, such as a lipophilic fluid or a polar hydrophobic fluid, serves to further isolate the particle-bound analyte from remaining components of the sample, which tend to remain in the water-miscible aqueous phase.

The “water-immiscible fluid” is a liquid or semisolid fluid that phase-separates when diluted with an equal part of water; preferably, the fluid phase-separates when diluted 2:1, 4:1, or 10:1 with water. More preferably, the water-immiscible fluid is substantially fully immiscible with water; it is preferably immiscible with lower alcohols as well. Examples of suitable water-immiscible fluids include lipophilic fluids such as waxes, preferably liquid waxes such as Chill-Out™ 14 wax (MJ Research), and oils, such as mineral oil, paraffin oil, or silicone, fluorosilicone, or fluorocarbon oils. Semisolid waxes may also be used, as long as the external force applied is sufficient to move the solid phase carrier through the medium; heat may be applied to reduce viscosity. In general, waxes and oils that are liquid at room temperature are preferred. Also suitable are, for example, hydrocarbon solvents such as toluene, hexane, or octane, and polar hydrophobic solvents such as 1,4-dioxane, acetonitrile, tert-butanol or higher (up to about C12) alcohols or acetates, cyclohexanone, or t-butyl methyl ether. If a polar hydrophilic solvent is employed, the water-miscible liquid reagents employed in the device preferably do not include substantial amounts of lower alcohols. Preferably, the water-immiscible fluid has a low vapor pressure and a specific gravity less than that of water. In selected embodiments, the water-immiscible fluid is an oil, such as mineral oil.

The particles may then be moved into chamber 38 (second wash chamber), which, in some embodiments, contains an alcohol or water/alcohol solution, such as ethanol or aqueous ethanol. In alternative embodiments, particularly in situations where traces of alcohol in processing chamber 36 are to be avoided, chamber 38 may be bypassed.

In selected embodiments, the particles are moved, via the external force, either after washing in chamber 38 or in lieu of washing in chamber 38, into chamber 40, situated above the channel region, which contains no fluid. The particles may be dried therein using air, including pressurized air, and/or heat. The particles are then moved back into second channel region 28 containing water-immiscible fluid 62. If desired, this movement may be facilitated by dispensing further water-immiscible fluid into chamber 40 after the particles have been dried; the further water-immiscible fluid may be dispensed from storage compartment 56 via channel region 28, or it may be dispensed from a separate storage compartment (not shown) associated with chamber 40.

With the exception of the possible use of chamber 40 for air-drying, the particles preferably remain in contact with liquid throughout their movement through the device.

In embodiments in which chamber 38 is bypassed, it may nonetheless contain an aqueous or aqueous/alcohol solution, in order to reduce the amount of water-immiscible fluid required to fill channel region 28; alternatively, additional water-immiscible fluid may be employed, effective to fill chamber 38 and channel region 28. In this case, storage compartment 54 could contain water-immiscible fluid.

Subsequent to washing in chamber 38 and/or drying in chamber 40, the particles are moved through water-immiscible fluid 62 into elution/processing chamber (third chamber) 36. In one embodiment, this region of channel region 28, just upstream of chamber 36, may contain a narrowing region 64, as described above. In addition to helping control fluid flow as described above, this narrowing may serve to reduce clumping of the particles as they prepare to contact the liquid reagent in chamber 36. In the embodiment shown in FIG. 3, the cross-sectional width of the device is also less in the area of process chamber 36, which may serve to improve heat transfer in this region.

Preferably, the processing chamber 36, together with the channel 23 leading to the chamber, contains a precisely known amount of reagent solution 74 as described above. The reagent solution, in one embodiment, comprises an elution buffer, which is effective to remove the bound isolated analyte from the particles. In some cases, heat may also be applied; e.g. to release hybridized nucleic acids from a probe attached to the particles. Other reagents, such as linkage cleaving reagents, including enzymes, may also be included as needed to facilitate release of the bound analyte from the particles.

With reference to FIGS. 5A-5C, a method of using the device will now be described. A device as shown in the drawings is provided, and a sample is introduced into the first chamber (84) via the sample entry port (82) and conduit (86). In one embodiment, a cap on the sample entry port is removed, and sample is introduced into the opening. The cap is replaced and the sample is drawn into the first chamber, for example, by gravity (depending on relative placement of the entry port, conduit and chamber) or by a pulse of air by a piston contained in the cap. In one embodiment, a reagent in dried or lyophilized form is contained in the first chamber, and is solubilized by the liquid sample, and further solubilized by fluid in the storage chamber associated with the first chamber when the fluid is dispensed into the first chamber. After the sample is introduced into the device, the fluid in the storage chamber associated with the first chamber is dispensed, typically by applying pressure to the storage chamber causing it to break at a predetermined position and fluid to flow into the associated chamber. Burstable storage chambers are described, for example, in U.S. Patent Application Publication No. 2012/0117811, which is incorporated by reference herein. Concurrent with fluid being dispensed into the first chamber, the fluid in the storage compartments associated with the second chamber, the processing chamber, and if present, any other chambers (such as chamber 94 in FIGS. 5A-5C). In a desired embodiment, the volume of fluid in a storage compartment associated with a chamber is selected to achieve a desired goal or outcome. For example, in one embodiment, the capacity of the first chamber is larger than the volume of fluid in the storage compartment associated with the first chamber, so that fluid in the first chamber does not flow into the channel that connects the first chamber with an adjacent, downstream chamber (for example, channel 92 in FIGS. 5A-5B). In another embodiment, the volume of fluid in a storage compartment associated with a chamber is larger than the capacity of the chamber, so that by design fluid in the storage compartment overfills the associated chamber and flows into a channel or conduit in the fluid flow path of the device. By way of example, in one embodiment, the volume of fluid in the storage compartment associated with the processing chamber (such as chamber 110 in FIGS. 5A-5B) is greater than the capacity of the processing chamber. Fluid in the storage compartment associated with the processing chamber fills to capacity the processing chamber and flows into the conduit upstream of the processing chamber (e.g., conduit 108 in FIGS. 5A-5B).

After fluid is introduced into each of the chambers in the device, the storage compartment filled with the immiscible fluid is opened, to dispense its contents into the device. In the device embodiment of FIGS. 5A-5B, the immiscible fluid flow via port 132 into conduit 136. Fluid in the processing chamber that has overflowed into conduit 108 is displaced by the immiscible fluid and pushed into an overflow chamber, such as the lower portion 102 of chamber 100 in the device of FIGS. 5A-5B. As can be appreciated, this approach permits precise control over the amount of fluid in the processing chamber. The amount of immiscible fluid in the storage compartment is sufficient flow into the channel of the flow path in the cartridge. For example, the immiscible fluid fills the lower portion 102 of chamber 100, and flows in the channel upstream of chamber 100 (e.g., channel 96 in the device of FIGS. 5A-5B). Once the immiscible fluid is dispensed, a series of fluid/immiscible fluid interfaces in the device are defined. For example, a first fluid/immiscible fluid interface exists at the junction of processing chamber (110 in FIGS. 5A-5B) and the channel upstream of the processing chamber (channel 108 in FIGS. 5A-5B). Another fluid/immiscible fluid interface is created at the junction between wash chamber 94 and the channel leading into the chamber (channel 96 in FIGS. 5A-5B). In one embodiment, another fluid/immiscible fluid interface is created at the junction between wash chamber 90 and the channel leading into the chamber (channel 111 in FIGS. 5A-5B). After the fluids are introduced into the device, and when the solid carrier particle/analyte complex(es) is/are moved from the first chamber to downstream subsequent chambers, the fluid/immiscible fluid interfaces remain stationary.

In another embodiment, the method is carried out, in accordance with the same basic principles described above, using a device such as illustrated in FIGS. 6-10. Preferably, the biological sample is first introduced into the first chamber (32) via the inlet port (66). Depending on the nature of the sample, it may be pretreated in various ways, e.g. by dilution with a standard buffer, if necessary. Liquid reagents are introduced into the chambers from the associated storage compartments, preferably in a preselected and automated sequence. The selection of liquid reagents and the sequence in which they are added will depend on the process to be carried out within the device. Chambers may include, for example, reagents for isolation, separation, modification, labeling, and/or detection of analytes. Reagents may be added simultaneously and/or in sequence.

In a preferred embodiment, a lysis buffer, effective to lyse cells in a biological sample, is introduced, from storage compartment 48, into first chamber 32 (FIG. 9; diagonal hatching). The amount of buffer may be such that chamber 32 is slightly underfilled; in this case, the introduction of wash buffer 60, below, completes the filling of chamber 32.

Subsequently, in a preferred process sequence, a wash buffer 60 is introduced, from storage compartment 50, into second chamber 34. As shown in FIG. 9, storage compartment 50 delivers a volume of liquid reagent 60 (horizontal hatching), in this case wash buffer, that is greater than the volume of second chamber 34, such that a portion of the liquid reagent 60 flows into the section of second channel region 28 that is immediately adjacent/downstream of chamber 34. As noted above, conduit 80 may be connected directly to the second chamber; or to the first channel, preferably adjacent to the first chamber; or to a region of the first chamber immediately adjacent the first channel. Thus, a small amount of reagent 60 may also enter the top portion of first chamber 32 (not shown in FIG. 9). Preferably, at the downstream end, water-miscible reagent (e.g. wash buffer) 60 extends to include the intersection of second channel region 28 with conduit 58 (but does not reach the intersection with conduit 39).

First channel 24 between the first and second chambers is preferably of a length and narrowness, relative to the chambers, to minimize mixing of fluids between the two chambers. In addition, the second channel region is in communication with the first channel and first cavity only via the second cavity. Thus, preferably, minimal fluid from the first chamber (e.g. lysis buffer) enters the first channel 24, even less enters the second chamber 28, and virtually none contacts the second channel region, which will eventually contain a zone 62 of water-immiscible fluid (FIG. 10).

As noted above, this design has advantages such as the following. It has been found that, when particles containing bound analyte freshly extracted from a lysis mixture in chamber 38 are washed in wash chamber 40 prior to being introduced to water-immiscible fluid in flow path 32, there is less tendency for the particles to clump and/or to stick to the walls of the chamber(s) and flow path(s), as compared to when particles containing bound analyte freshly extracted from lysis mixture in chamber 38 are introduced to the water-immiscible fluid.

Simultaneously with, subsequent to, or prior to addition of the lysis buffer and the wash buffer 60, an elution and/or reaction buffer (FIG. 10; stippling), in a preferred embodiment, is added to third chamber (process chamber) 36 from storage compartment 52. Preferably, in addition, an alcohol/water or aqueous wash solution (FIG. 10; broken diagonal hatching) is added to chamber 38 from storage compartment 54, either simultaneously with, subsequent to, or prior to addition of the wash buffer 60.

Subsequent to the placement of the water-miscible fluids in their respective chambers, a water-immiscible fluid, such as described above, is introduced from storage compartment 56, via channel 58, such that the water-immiscible fluid (vertical hatching in FIG. 10) extends from the junction of conduit 58 with the second channel region to include at least first intersection 25 with third channel 23. Accordingly, fluid interfaces (between water-miscible and water-immiscible fluid) are created at 90 and 92, with the second aqueous reagent and third aqueous reagent, respectively, as shown in FIG. 10. In a preferred embodiment, where fourth chamber 38 and its associated conduit 39 also contain a water-immiscible reagent, a further fluid interface is formed at 164 (FIG. 10). Preferably, all of these fluid interfaces, formed when the fluids are dispensed into the chambers and channels in accordance with the disclosed method, remain essentially stationary when the solid carrier particles are moved through the device, in a manner to be described below.

As noted above, the third storage compartment 52 preferably delivers a volume of liquid reagent that is greater than the volume of third chamber 36 (processing chamber), for the purpose of precisely defining the amount of fluid in the chamber. Thus, delivery of a precise volume to chamber 36 is achieved by first overfilling the chamber 36 with the liquid reagent (e.g. elution and/or amplification buffer), and preferably also overfilling adjacent channel 23, such that an excess amount of reagent enters channel region 28. Subsequently, a water-immiscible fluid is introduced from storage compartment 56, via channel 58, such that the water-immiscible fluid displaces the excess solution volume from channel region 28, e.g. to a drain within chamber 40, thereby precisely defining the volume of fluid in chamber 36 and adjacent channel 23 to their known machined volume, terminating at interface 162.

At some stage before, during or after the addition of fluid reagents, a solid carrier, such as a plurality of affinity-treated particles (not shown in the Figures) is added to first chamber 32. The device may also be provided to the user, in a preferred embodiment, with the particles already in place in the first chamber. At least a plurality and preferably all of the particles comprise an attached specific binding member, as described above, which is effective to specifically and reversibly bind the target analyte(s); e.g. by specific antibody-antigen binding, by hybridization, by ionic or hydrogen bonding, or other chemical interaction. The binding moiety may be, for example, a nucleic acid probe sequence, effective to hybridize to a target nucleic acid sequence, or an antibody or functional fragment thereof, effective to bind a target protein or other analyte. Any binding moiety of any desired specificity may be used.

The particle-bound analyte is then exposed to the various liquid reagents within the device by a process in which the particles are moved, by virtue of an externally applied force, though the chambers and channels. Thus, following the disposition of fluids into the respective chambers and channels, to give the arrangement shown, for example, in FIG. 10, there is preferably minimal transport of fluid within the device. Preferably, all of the fluid interfaces, formed when the fluids are dispensed into the chambers and channels in accordance with the disclosed method, remain essentially stationary when the solid carrier particles are moved through the device.

Preferably, the particles are paramagnetic particles, such that they can be moved through the chambers and channels via an externally applied magnetic force. However, other means of moving the particles via an externally applied force can be used, including air pressure, vacuum, centrifugal force, or electrical fields for charged molecules or particles.

In a preferred embodiment, the sample is admixed with lysis buffer and affinity-treated particles in first chamber 32, for a sufficient time, at a sufficient temperature, and with sufficient agitation to lyse cells and allow the target analyte, if present, to bind to the affinity-treated particles. As noted above, the external sides of the device corresponding to first chamber 32 are accessible to a heat source if required, and mixing elements such as stir bars, stir particles, or “washboard” surfaces are preferably provided within the chamber. Mixing may also be facilitated by moving the particles within the chamber by the above-referenced externally applied force.

Following lysis and binding, in the exemplified process, the particles are transported, by application of the external force, to the second chamber 34, containing, in the present scenario, a wash buffer, such as a tris HCl buffer or PBS. Accordingly, the particles are moved through first channel 24, which has a narrow profile relative to the chambers and which is, preferably, largely filled with wash buffer 60, thus minimizing transfer of fluid from the first chamber to the second chamber. The device channels 24, 28, 23, and 29 are generally of narrow diameter for this purpose, but are of sufficient diameter to allow a plurality of moderately clumped particles to pass therethrough. Commercially available magnetic particles employed in the biomedical field range in size from less than 1 micron up to 100 microns, most commonly in the size range of 2-10 micron; preferably, the particles employed herein are about 1-3 microns in size. Thus, the channels through which the particles pass are preferably about 5 mm or less, more preferably about 2.5 mm or less, and most preferably about 1 mm or less in diameter or width. In some embodiments, the channels may be about 0.5 mm, about 0.3 mm, or about 0.1 mm or less in diameter or width. Preferably, the channels are at least 0.1 mm in diameter or width, and more preferably at least 0.5 mm in diameter or width.

Upon entering the second chamber 34, the solution therein and/or the particles may be further agitated, using one or more agitation strategies as described for the first chamber.

The particles are then moved into the zone of water-immiscible fluid 62 present in the second channel region 28. As described in U.S. Patent Appn. Pubn. No. 2009/0246782, which is incorporated herein by reference, movement of the carrier particles into the water-immiscible fluid, such as a lipophilic fluid or a polar hydrophobic fluid, serves to further isolate the particle-bound analyte from remaining components of the sample, which tend to remain in the water-miscible aqueous phase.

The particles may then be moved into chamber 38 (second wash chamber), which, in some embodiments, contains an alcohol or water/alcohol solution, such as ethanol or aqueous ethanol. In alternative embodiments, particularly in situations where traces of alcohol in process chamber 36 are to be avoided, chamber 38 may be bypassed. In embodiments in which chamber 38 is bypassed, it may nonetheless contain an aqueous or aqueous/alcohol solution.

In selected embodiments, the particles are moved, via the external force, either after washing in chamber 38 or in lieu of washing in chamber 38, into chamber 40, preferably situated above the second channel region, which, at this stage, either contains no fluid or contains a quantity of water-immiscible fluid sufficient to fill only a portion of the chamber, as shown in FIG. 9. As noted above, chamber 40 may contain a plurality of compartments, for this purpose, having different depths, as depicted in FIG. 6A.

The particles may be dried within an empty region of chamber 40, using e.g. air, including pressurized air, and/or heat.

The particles are then moved back into the downstream portion of second channel region 28, containing water-immiscible fluid 62. Preferably, this movement is facilitated by the presence of water-immiscible fluid in chamber 40; the particles may be moved into the fluid already present, or additional such fluid may be dispensed into the chamber after the particles have been dried, as shown e.g. in FIG. 10. The further water-immiscible fluid may be dispensed from storage compartment 56 via channel 28, or it may be dispensed from a separate storage compartment (not shown) associated with chamber 40.

With the exception of the possible use of chamber 40 for air-drying, the particles preferably remain in contact with either water-miscible or water-immiscible fluid throughout their movement through the device.

Subsequent to washing in chamber 38 and/or drying in chamber 40, the particles are moved through water-immiscible fluid 62 into channel 23, containing elution reagent, and thence into elution/process chamber (third chamber) 36.

In one embodiment, third channel 23 has a particularly narrow profile, as shown in the Figures. This narrowing may serve to reduce clumping of the particles as they prepare to contact the liquid reagent in chamber 36. In the embodiment shown in FIG. 3, the cross-sectional width of the device is also less in the area of processing chamber 36, which may serve to improve heat transfer in this region.

Preferably, the processing chamber 36 contains a precisely known amount of reagent solution 74. In a manner similar to that described above for the horizontal embodiment, a slight excess of elution and/or reaction buffer (FIG. 10; stippling) may be introduced into third chamber 36 and third channel 23, during introduction of the water-miscible reagents, such that some elution and/or reaction buffer enters second channel region 28; subsequent introduction of the water-immiscible fluid into second channel region 28 can serve to remove this excess, when creating interface 162, thus providing a precise volume within third chamber (process chamber) 36 and third channel 23.

The third reagent solution, in a preferred embodiment, comprises an elution buffer, which is effective to remove the bound isolated analyte from the particles. In some cases, heat may also be applied; e.g. to release hybridized nucleic acids from a probe attached to the particles. Other reagents, such as linkage cleaving reagents, including enzymes, may also be included as needed to facilitate release of the bound analyte from the particles. Diversion channel 96 is preferably provided for segregation of the solid phase carrier from the solution containing released analyte.

C. Processing of Sample

In one embodiment, the processing chamber or elution chamber 36 is used for amplification and detection of a target nucleic acid. In this embodiment, the elution buffer may also contain amplification reagents, e.g. primers, labeled probes, nucleotides, and the necessary enzymes, as known in the art. Alternatively, amplification reagents (or other chemical process reagents) may be included in wax-coated lyophilized form, as shown at 72 in FIG. 3; heating of the chamber at a preselected time releases the reagents.

Such amplification may use any amplification method known in the art; examples include, but are not limited to, PCR, RT (real time)-PCR, RT (reverse transcriptase)-PCR, and isothermal techniques such as nucleic acid sequence based amplification (NASBA), transcription mediated amplification (TMA), strand displacement amplification (SDA), ligase chain reaction (LCR), and helicase dependent amplification (SDA).

Although nucleic acid isolation and amplification is exemplified here, the device and its use are not limited to specific chemical processes or analytes. In some preferred embodiments, for example, the device is used for protein isolation and detection.

The chamber 36 is also provided with optically transparent windows 70 such that optical signals, typically indicative of the presence of an analyte, can be detected. In one embodiment, RT-PCR is carried out within chamber 36 and monitored via windows 70. In other embodiments, results of immunoassays or colorimetric or fluorimetric assays, e.g. for protein detection, can be observed via windows 70.

### IV. Automated System

As noted above, the cartridge device is designed for automated use within a instrument that may hold one or a plurality of such devices. The cartridge is inserted into the instrument after loading of the sample, and fluids are dispensed from the reagent storage compartments, in the appropriate order, in automated fashion. The particles are moved through the device by an externally applied force, preferably a magnetic force, also in automated fashion.

The instrument is supplied with heating elements capable of carrying out thermal cycling processes and optics and software for analyzing and reporting assay results. In one embodiment, a mechanical stage is used to move the cartridge device(s) to and from e.g. heating elements, magnetic bead mover(s), and thermal cycling stations as needed. In one exemplary design, the instrument includes a cartridge loading and unloading station, with the capacity for several cartridges; a sample processing station, which includes stations dedicated to liquid dispensing, mixing, particle moving, and heating; and a thermal cycling station, supplied with an optical detection station.

For maximum ease of use, with minimal required user manipulation, the cartridge device is preferably provided with the appropriately functionalized particles, designed to specifically bind a particular analyte species, within first chamber, to which the sample is added. Suitable reagents are provided in the various sealed storage compartments for carrying out the isolation and, preferably, the detection and/or quantification of the specified analyte. In one embodiment, isolation, amplification and detection of specific nucleic acids, characteristic of an analyte, are carried out within the device.

The cartridge device is preferably labeled, most preferably by bar coding, designating e.g. the analyte, the analysis protocol, and the lot number and expiration date of the cartridge and contents. Preferably, the cartridge contents are storage stable under standard refrigeration or, more preferably, at room temperature, for a year or longer, preferably 18 months or longer.

### V. Analytes

The use of the device is not limited to any particular analyte, group of analytes, or sample types. As known in the art, disease can be diagnosed and monitored by detection of nucleic acids and/or proteins associated with disease pathogens, and/or by quantitation of endogenous biological markers. Cell counts and other types of body fluid analysis can also be used to monitor patient health. As noted above, the cartridge device and instrument are expected to be particular useful in geographical areas that have reduced access to technical training and to expensive analytical equipment. In particular, there is an increasing need for low-cost, rapid and reliable diagnosis and monitoring of diseases such as HIV, tuberculosis, and pertussis in the developing world.

Accordingly, the cartridge device can be supplied with particles treated to selectively bind to such a nucleic acid or protein, and assay reagents, which may include, for example, labeled antibodies, nucleic acid amplification reagents, and/or labeled probes, can be supplied in one or more process chambers within the device.

### VI. Examples

The following example is illustrative in nature and in no way intended to be limiting.

### Example 1

### Purification and Amplification of HIV-1 RNA from Plasma

Quantitative measurement of HIV-1 is important for monitoring disease progression and evaluating antiretroviral drug therapy outcome. Viral load measurement is technically demanding, due to the relatively low viral copy number and abundance of PCR inhibitors in samples derived from human blood. In accordance with the device and methods described herein, the level of HIV-1 RNA in a blood sample is quantitated in an automated manner.

The first chamber of the cartridge device is provided with RNA-binding paramagnetic particles, e.g. a 5 μL aliquot of Ambion® MagMax™ Total RNA magnetic beads. A 50 μL sample of plasma suspected of containing HIV-1 virus is added to the first chamber. The cartridge is then placed into the cartridge loading station of an instrument also having a sample processing station, which includes stations dedicated to liquid dispensing, mixing, particle moving, and heating; and a thermal cycling station, supplied with an optical detection station.

To the first chamber of the device is then automatically dispensed, from the first storage compartment, an aqueous lysis solution sufficient to fill the first chamber, containing e.g. lysis and binding reagents in the following proportions: 200:1:5:200 Ambion Lysis/Binding solution concentrate, carrier RNA, Binding Enhancer (all supplied by Applied Biosystems; Foster City, Calif.), and isopropyl alcohol.

Wash buffer (e.g. 100 mM Tris HCl, 150 mM NaCl or LiCl, and 50 mM sodium citrate) is added to the second (wash) chamber, from the second storage compartment, sufficient to fill the second chamber and to displace the lysis/binding solution from the first channel.

PCR/elution buffer, containing primers, probes, and other reagents effective to amplify the target HIV-1 RNA, is dispensed from the third storage compartment into the third (process) chamber and the associated elution channel of the cartridge. The buffer contains, for example, components of the Abbott RealTime HIV-1 Amplification Reagent Kit (Abbott Molecular, Des Plaines, Ill.), with the addition of 0.2 mg/ml bovine serum albumin, 150 mM trehalose, and 0.2% Tween 20.

The elution buffer, and the channel between the wash and process chambers, are then overlaid with a water-immiscible fluid, such as mineral oil or Chill-Out™ liquid wax (Biorad Laboratories; Hercules, Calif.), automatically dispensed from an onboard storage compartment. The water-immiscible fluid displaces the PCR/elution buffer that is present above a predetermined point in the elution channel, and the displaced excess flows to an upstream chamber within the second channel region.

The contents of the first (lysis) chamber are mixed for ˜4 minutes, by magnetic dispersal of the particles and/or a magnetic stirring element. The automated system aggregates the particles in the first chamber for ˜2 minutes, using an external magnet, and then moves the aggregate from the lysis buffer to the wash buffer, then through the water-immiscible fluid, and to the elution buffer.

The PCR/elution buffer containing the particles is heated to 55° C. for 10 minutes to elute the RNA from the particles, which are then magnetically aggregated.

The cartridge device is then transferred to a thermal cycling station within the instrument, where HIV-1 viral load quantification is performed. Progress of RT-PCR amplification is monitored, and the presence and/or amount of HIV-1 is reported. A high PCR efficiency is indicative that carryover of inhibitors from lysis and wash solutions in the device is minimal.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A sample processing device, comprising:
a rigid body having a first side and a second side, and defining at least:
a first cavity, a second cavity, and a third cavity, wherein the first, second and third cavities are associated with first, second, and third storage compartments, respectively, each containing a water-miscible liquid reagent,
a first channel, connecting the first cavity and the second cavity, and
a second channel region, in fluid communication with and downstream of the second cavity, and connected to the third cavity via a third channel, at a first intersection,
wherein said second channel region is associated with a storage compartment containing a water-immiscible fluid,
a wall member secured to at least a portion of the first side of the rigid body, said wall member disposed over the first cavity, the second cavity, and the third cavity, thereby defining a first chamber, a second chamber, and a third chamber; and
an inlet port in communication with the first chamber.

2. The device of claim 1, wherein said second channel region is in communication with said first channel and first cavity only via said second cavity.

3. The device of claim 1, wherein said storage compartment containing a water-immiscible fluid contains a volume of said fluid that is sufficient, when dispensed to said second channel region from said storage compartment, to produce a continuous layer of said fluid within the second channel region that includes said first intersection.

4. The device of claim 1, wherein said wall member comprises a plurality of blister regions defining said liquid reagent storage compartments.

5. The device of claim 1, wherein the water-miscible liquid reagent in each of the first, second and third storage compartments is selected from an aqueous buffer, a water-containing lysis buffer, a water-based salt solution, and an elution medium.

6. The device of claim 1, wherein said second storage compartment contains a volume of liquid reagent that is greater than the combined volume of the second chamber and any intermediary conduit.

7. The device of claim 6, wherein said second storage compartment contains at least a volume of liquid reagent effective to fill said second chamber, said first channel and any intermediary conduit.

8. The device of claim 1, wherein the third storage compartment contains a volume of liquid reagent that is greater than the combined volume of the third chamber, the third channel and any intermediary conduit.

9. The device of claim 1, wherein the third chamber comprises an optically transparent window which makes up a portion of the exterior surface of the rigid body.

10. A method for extracting an analyte of interest from a sample, comprising:
(i) providing a device comprising: a first chamber, containing a solid phase carrier and comprising a sample port, a second chamber, and a third chamber which is a process chamber,
a first channel, connecting the first chamber and the second chamber, and
a second channel region, in fluid communication with and downstream of the second chamber, and connected to the third chamber via a third channel, at a first intersection;
(ii) introducing into said first chamber, a volume of a first aqueous reagent and a sample, wherein said solid phase carrier is effective to selectively bind an analyte if present in said sample;
(iii) introducing a volume of a second aqueous reagent into the second chamber, effective to fill the second chamber and at least a portion of said first channel; and introducing a volume of a third aqueous reagent into said third chamber and third channel; (iv) introducing a volume of water-immiscible fluid into said second channel region, such that said water-immiscible fluid forms a contiguous zone of fluid within said second channel region that includes said first intersection, and forms first and second fluid interfaces, respectively, with said second aqueous reagent and with said third aqueous reagent; and
(vi) with an externally applied force, moving the solid phase carrier, sequentially, into the aqueous reagent in the second chamber, into the water-immiscible fluid, and into the third aqueous reagent in the third channel and processing chamber, whereby said moving transfers the solid phase carrier and associated analyte of interest, thereby extracting the analyte of interest from the sample.

11. The method of claim 10 wherein said fluid interfaces remain essentially stationary during said moving.

12. The method of claim 10, wherein said device further comprises a drying chamber, which is connected to said second channel region at a point at or upstream of said first intersection, and the method further comprises, prior to moving the solid phase carrier into the third channel and processing chamber, moving the solid phase carrier into said drying chamber, and subsequently filling at least the portion of the drying chamber containing the solid phase carrier with the water-immiscible fluid.

13. The method of claim 10, wherein the volume of reagent introduced into the process chamber is greater than the volume of the process chamber, such that an excess portion of the process chamber reagent flows into a channel in communication with and upstream of the process chamber;
and wherein said introducing of said water-immiscible fluid is effective to displace said excess portion of the process chamber reagent at a predetermined location, thereby achieving a contiguous volume of process chamber reagent within the device that is known and precise.

14. The method of claim 10, wherein said first channel includes a constriction region having a dimension, and a divider having a first height;
said second channel region includes a divider having a second height which is greater than said first height;
the volume of aqueous reagent introduced into said second chamber is effective to fill said second chamber and said first channel, to a level above said first divider but below said second divider;
and the combined volume of aqueous reagent introduced into said first and second chambers is effective to fill said first and second chambers and said first channel, to a level above said first divider but below said second divider.

15. The method of claim 14, wherein the solid phase carrier comprises a plurality of solid carrier particles, and the number of particles in the plurality of solid carrier particles, the size of each particle in the plurality of solid carrier particles, and the dimension of the constriction region are selected such that the plurality of solid carrier particles individually and collectively can pass through the constriction region;
and wherein the constriction region reduces transfer of aqueous reagent via the first channel between the first and second chambers.
